(19) United States
(12) Patent Application Publication
(10) Pub. No.: US 2025/0282835 A1
(43) Pub. Date: Sep. 11, 2025

(54) IMPROVED VARIANTS OF MONOMERIC SCARLET RED FLUORESCENT PROTEIN

(71) Applicant: UNIVERSITEIT VAN AMSTERDAM, WX Amsterdam (NL)

(72) Inventors: Theodorus Wilhelmus Johannes GADELLA, Amsterdam (NL); Laura VAN WEEREN, Amsterdam (NL)

(73) Assignee: UNIVERSITEIT VAN AMSTERDAM, WX Amsterdam (NL)

(21) Appl. No.: 18/564,692

(22) PCT Filed: Jun. 2, 2022
(86) PCT No.: PCT/EP2022/065029

§ 371 Date: Nov. 28, 2023

(30) Foreign Application Priority Data
Jun. 3, 2021 (EP) 21177552.3

(51) Int. Cl. C07K 14/435

(52) U.S. Cl. CPC C07K 14/43595

(57) ABSTRACT

A monomeric Scarlet Red Fluorescent protein (mScarlet RFP) includes at least amino acid substitutions V196I and/or G220A when compared to the amino acid sequence of synthetically designed mScarlet RFP. The V196I and/or G220A variants may include one or more further amino acid substitutions.

### REFERENCE TO AN ELECTRONIC SEQUENCE LISTING

The present application contains a Sequence Listing that has been submitted electronically and is hereby incorporated by reference in its entirety. The electronic Sequence Listing is named Sequence_Listing_ST25, was created on May 16, 2024, and is 59,606 bytes in size.

The present invention relates to variants of a monomeric Scarlet Red Fluorescent protein having enhanced intrinsic brightness and maturation.

Fluorescent proteins (FPs) are indispensable in biological research. They can be used as fusion tag, and spectral variants of FP's can be applied in Förster resonance energy transfer (FRET) based biosensors to probe molecular interactions, conformational changes, and metabolite concentrations within living cells. The use of red FPs (RFPs) as the donor or acceptor or acceptor in FRET pairs has an advantage over FRET pairs with cyan FPs, and yellow FPs, because the RFPs allow excitation at longer wavelengths, which is less harmful or toxic to biological samples, induces less autofluorescence, and enables multiplexing of several FRET sensors.

Monomeric Scarlet red fluorescent protein (mScarlet RFP) is a synthetic, monomeric red fluorescent protein that has been developed from other naturally occurring RFPs and chromo proteins. Monomeric Scarlet RFP, herein after referred to as mScarlet RFP or mScarlet. has the amino acid sequence of SEQ ID NO: 1 and is encoded by the nucleic acid sequence of SEQ ID NO: 2. A variant of mScarlet with the single amino acid substitution T74I was also described, hereinafter referred to as mScarlet-I RFP or mScarlet-I. Although mScarlet and its variant mScarlet-I are synthetic RFP that have higher intrinsic brightness, quantum yield and fluorescence lifetime than other mRFPs, mScarlet has a low maturation efficiency that affects the overall cellular brightness, making it less suitable for biological imaging. The single amino acid substitution T74I that was found in the variant mScarlet-I resulted in a marked maturation acceleration in cells relative to mScarlet, albeit accompanied by a decrease in fluorescence quantum yield and fluorescence lifetime due to a lower intrinsic brightness. Hence there is a need for variants of mScarlet RFP that combine the high intrinsic brightness of mScarlet with the enhanced maturation efficiency/speed of mScarlet-I. In the search for such variants, mutants were generated of which both the apparent cellular brightness in cells 24 h after expression and the fluorescence lifetime were monitored. Since the fluorescence lifetime is proportional to the fluorescence quantum yield and the extinction coefficients of the diverse mScarlet variants are more or less invariant, the fluorescence lifetime is proportional to the intrinsic brightness of the mScarlet variant. The cellular brightness, which is relevant for biological applications is determined by the product of the intrinsic brightness and the extent of maturation of the variant. By simultaneous screening for both parameters, the effect of individual mutations on both intrinsic and cellular brightness can be discerned. It was found that introducing the amino acid substitution G220A resulted in an increased maturation extent and cellular brightness, yet at the cost of intrinsic brightness, but when combined with V196I, an increase of intrinsic and a decrease in cellular brightness could be observed (see FIG. 7), both in the absence and presence of the T74I mutation.

This indicated that these two mutations markedly influence the balance between maturation and intrinsic brightness, thereby increasing the overall cellular brightness relative to the cellular brightness of mScarlet (SEQ ID NO:1).

The present invention provides for variants of mScarlet, wherein the variant comprises at least amino acid substitutions V196I and/or G220A, and optionally amino acid substitution T74I, when compared to the amino acid sequence of the mScarlet RFP depicted in SEQ ID NO:1. It was found that by introducing the amino acid substitution G220A, or a combination of amino acid substitutions G220A and V196I into the amino acid sequence of mScarlet, variants of mScarlet could be obtained that have enhanced intrinsic and cellular brightness. When combined with the substitution T74I the resulting variants not only showed enhanced brightness but an increase in maturation relative to mScarlet.

The V196I and/or G220A variants of the present invention may comprise one or more further amino acid substitutions. Preferred additional amino substitutions are E219V or the combination of T107S and G156V. In particular the combination of E219V, T107S and G156V in addition to V196I and/or G220A, and optionally T74I, was found to have a beneficial effect on the intrinsic brightness and maturation of the variants of the invention.

The properties of the variants described here before can be further enhanced by additionally introducing amino acid substitution Y84W alone, or in combination with Y194F. These substitutions were found to be beneficial for the intrinsic brightness, and consequently the overall cellular brightness of the proteins.

In addition to the amino acid substitutions described here before, the improved variants of mScarlet RFP may comprise further amino acid substitutions that stabilize the configuration. In particular substitutions T109A, and K183R, and optionally K48R were found to stabilize the configuration of improved mScarlet variants without the T74I substitution. The substitutions N99I, A105T, T128G, K140R, and optionally K93R were found to stabilize improved mScarlet variants that comprises the T74I substitution.

The amino acid substitutions referred to in the invention are all relative to the amino acid sequence of SEQ ID NO: 1 unless otherwise indicated. The amino acid residues are identified by the one-letter code. Preferably, variants of the present invention comprise at least amino acid substitutions V196I, G220A, E219V, T107S and G156V, and optionally T74I. More preferably, the variant comprises at least amino acid substitutions V196I, G220A, E219V, T107S, G156V, Y84W, and optionally T74I, or the variant comprises at least amino acid substitutions V196I, G220A, E219V, T107S, G156V, Y84W, Y194F, and optionally T74I. Of particular interest are the variants comprising at least the amino acid substitutions K48R, Y84W, T107S, T109A, G156V, K183R, Y194F, V196I, E219V, and G220A, and the variants comprising at least amino acid substitutions T74I, Y84W, N99I, A105T, T107S, T128G, K140R, G156V, Y194F, V196I, E219V and G220A. The mScarlet variants of the invention have a sequence identity of at least 80%, preferably 85%, more preferably 86%, 87%, 89%, or 90% with the amino acid sequence of the parent mScarlet RFP depicted in SEQ ID NO: 1, as determined by the BLAST algorithm over the complete 232 amino acid sequence of mScarlet. Like the monomeric RFP mScarlet, the mScarlet variants of the invention are monomeric and have a higher intrinsic brightness, quantum yield and fluorescence lifetime than other monomeric RFPs.

The mutations Y84W, Y194F, V196I, G220A are facing internally in the beta-barrel of the mScarlet variants and are key to the improved properties of the mScarlet variants. Together these mutations form an interacting hydrophobic pocket inside the beta barrel, that is responsible for the increase in the properties of the variants. Without being bound by theory, the mutations T107S, G156V and E219V, which are all outside the beta barrel, were all found to apparently support/stabilize the internal mutations in the beta barrel of mScarlet variants according to the invention. In addition to the exterior mutations T107S, G156V and E219V, other (exterior) mutations K48R, T109A, K183R and were all found to apparently further support/stabilize the internal mutations in the beta-barrel of mScarlet variants without the T74I mutation, while a different set (exterior) mutations N99I, A105T, T128G, and K140R, were found to apparently further support/stabilize the internal mutations in the beta-barrel of mScarlet variants comprising the T74I mutations. Thus, in a preferred embodiment, the mScarlet variants of the invention comprise at least amino acid substitutions Y84W, Y194F, V196I, G220A, and optionally T74I relative to the amino acid sequence of mScarlet depicted in SEQ ID NO: 1. Preferably, the mScarlet variants of the invention comprise at least amino acid substitutions Y84W, Y194F, V196I, G220A, T107S, G156V and E219V, and optionally T74I, relative to the amino acid sequence of mScarlet depicted in SEQ ID NO: 1. More preferably, mScarlet variants without the T74I mutation comprise at least amino acid substitutions Y84W, Y194F, V196I, G220A, T107S, G156V, E219V, K48R, T109A, and K183R relative to the amino acid sequence of mScarlet depicted in SEQ ID NO: 1, and mScarlet variants with the T74I mutation comprise at least amino acid substitutions Y84W. Y194F, V196I, G220A, T107S, G156V, E219V, N99I, A105T, T128G, and K140R relative to the amino acid sequence of mScarlet depicted in SEQ ID NO: 1.

Furthermore, the mScarlet variants of the invention may have altered N and C termini, such as described e.g. in Valbuena et al. 2019. Thus, in a further embodiment, the mScarlet variants of the invention comprise amino acid substitutions and deletions ΔM1, V2M, S3D, K4S, G5T and M227S, D228G, E229G, L230S, ΔY231, ΔK232. These mutations lowered cytotoxicity of the proteins in bacteria.

In another embodiment, the mScarlet variants according to the invention are the variants comprising the amino acid sequence depicted in SEQ ID NO's 5, 7, 9, 11, 13, 15, 17, 19, 21, 23, 25, 27, 29, 31, 33, and 35, preferably SEQ ID NO's 23, 25, 27, 29, 31, 33, and 35, more preferably SEQ ID NO's 25, 29, 31, and 35. In a preferred embodiment the mScarlet variants of the invention consist of the amino acid sequence depicted in SEQ ID NO's 5, 7, 9, 11, 13, 15, 17, 19, 21, 23, 25, 27, 29, 31, 33, and 35, preferably SEQ ID NO's 23, 25, 27, 29, 31, 33, and 35, more preferably SEQ ID NO's 25, 29, 31, and 35. Highly preferred are the variants mScarlet-3 (SEQ ID NO: 31) and mScarlet-I3 (SEQ ID NO: 35) having altered N and C termini relative to the amino acid sequence of SEQ ID NO: 1 (mScarlet). The new N and C-termini were found to lower cytotoxicity in bacteria without having hardly any effect on brightness or maturation (efficiency/speed) of mScarlet3 or mScarlet-i3.

The mScarlet variants of the invention show an increase in intrinsic brightness, protein stability and maturation speed, resulting in an overall increase in cellular brightness. Therefore, these variants are especially useful for cellular imaging, in particular in prolonged imaging at normal light doses. All these variants show increased cellular brightness compared to mScarlet. Moreover, mScarlet3 and mScarlet-I3 are superior FRET acceptors.

Of particular interest are mScarlet variants without the T74I mutation comprising at least all 4 amino acid substitutions in the beta-barrel and at least the other (exterior) mutations K48R, T107S, T19A, G156V, K183R and E219V, and mScarlet variants comprising at least all 4 amino acid substitutions in the beta-barrel and at least the other (exterior) mutations N99I, A105T, T107S, T128G, K140R, G156V, E219V in addition to the T74I mutation. Particular preferred are the mScarlet variants comprising the amino acid sequence of SEQ ID NO: 31 or 35, more in particular consisting of the amino acid sequence depicted in SEQ ID NO: 31 or 35. These variants are the brightest red fluorescent proteins that have been obtained known to date worldwide. The improvements are manifold: an increased fluorescence quantum yield, an increased fluorescence lifetime, an increased pH stability, an increased maturation speed and -efficiency, a substantial increase in cellular brightness observed in mammalian cells, better performance as FRET acceptor, and reduced cytotoxicity in mammalian cells.

The mScarlet variants according to the invention can be obtained by site-directed and random mutagenesis using standard protocols, such as described in (Bindels et al., 2016). The mScarlet variants according to the invention can also be prepared via standard recombinant protein expression techniques. For this purpose, a nucleotide sequence encoding the respective mScarlet variant is inserted into an expression vector. Thus, in another embodiment, the invention provides for a nucleotide sequence encoding an mScarlet variant according to the invention, more preferably a nucleotide sequence encoding a variant having the amino depicted in one of the SEQ ID NO's one of the amino acid sequences 5, 7, 9, 11, 13, 15, 17, 19, 21, 23, 25, 27, 29, 31, 33, and 35, preferably SEQ ID NO's 23, 25, 27, 29, 31, 33, and 35, more preferably SEQ ID NO's 25, 29, 31, and 35, even more preferably SEQ ID NO's 31, and 35. Of particular interest are the nucleotide sequences of SEQ ID NO's 6, 8, 10, 12, 14, 16, 18, 20, 22, 24, 26, 28, 30, 32, 34, and 36, preferably SEQ ID NO's 24, 26, 28, 30, 32, 34, and 36, more preferably SEQ ID NO's 26, 30, 32, and 36, even more preferably SEQ ID NO's 32 and 36.

Suitable expression vectors are, amongst others, plasmids, cosmids, viruses and YAC's (Yeast Artificial Chromosomes) which comprise the necessary control regions for replication and expression. The expression vector can be brought to expression in a host cell, host tissue or host organism. Suitable host cells include but are not limited to bacteria, yeast cells, plant cells, insect cells and mammalian cells. Preferred host cells for this purpose are E. coli cells, HeLa cells and U2OS cells. Such expression techniques are well known in the art. (Sambrooke et al., Molecular Cloning: a Laboratory Manual, Cold Spring Harbor Laboratory Press, Cold Spring Harbor, 3^{rd} ed. c2001). Following expression, the expressed proteins can be isolated and purified from the medium using standard techniques well known in the art.

The mScarlet variants of the invention are suitable for use as fluorescent lifetime standards considering their high and monoexponentially fluorescence lifetime. The main application of mScarlet variants of the invention is the use of these FPs as superior fusion tag in cellular imaging for studying protein localization and dynamics in cells and tissues, and as FRET acceptor in FRET sensing/sensor applications. New FRET sensors may be built in which the new variants are used as FRET donor or acceptor to sense small metabolites, second messengers, protein-protein interactions, protein conformational changes and or protein post translational modifications in vitro, in cell-based assays or in situ. All these sensing applications can be used for new drug-screening methods and are for instance of interest for pharmaceutical companies for compound screening.

### LEGEND OF THE FIGURES

FIG. 1: Absorbance, excitation, and emission spectra of mScarlet3. For the fluorescence excitation spectrum, the emission was set at 620 nm, for the fluorescence emission spectrum excitation was set at 540 nm.

FIG. 2: Absorbance, excitation, and emission spectra of mScarlet-I3. For the fluorescence excitation spectrum, the emission was recorded at 620 nm, for the fluorescence emission spectrum excitation was at 540 nm

FIG. 3: TCSPC measurement of mScarlet3 fitted with a single exponential decay time of 3.96 ns. The weighted residuals show a perfect fit (χ²=1.18)

FIG. 4: pH dependence of the fluorescence intensity of mScarlet3, mScarlet-I3 and mScarlet

FIG. 5: Cellular brightness of RFPs in mammalian cells relative to mScarlet (larger bars are better).

FIG. 6: Cellular brightness of RFPs in mammalian cells after fixation relative to mScarlet (larger bars are better),

FIG. 7: Evolution path for mScarlet3 and mScarlet-I3 from mScarlet and mScarlet-I, respectively. The numbers correspond with the sequence IDs. The horizontal axis depicts the (modulation) fluorescence lifetime as determined in live cells and corresponds with intrinsic brightness (more to the right is better), and the vertical axis depicts the cellular brightness (higher is better). A: mScarlet-220A, B: mScarlet-219V220A, C: mScarlet-1961220A, A′: mScarlet-I-220A, B′: mScarlet-I-219V220A, C′: mScarlet-I-1961220A.

FIG. 8. Maturation extent of RFPs in mammalian cells relative to mScarlet-I (larger bars are better)

FIG. 9. Maturation speed of RFPs in mammalian cells (smaller bars are better)

FIG. 10. Alkaline denaturation speed of diverse purified RFPs at pH 13.5

FIG. 11: FRET spectra from HeLa cells expressing SYFP2 alone or SYFP2-RFP fusion proteins 43 h after transfection

FIG. 12: Quantification of FRET spectra from FIG. 10 (higher bars are better).

FIG. 13. FRET-FLIM results of HeLa cells expressing SYFP2 alone or SYFP2-RFP fusion proteins 48 h after transfection (lower bars indicate more FRET).

FIG. 14: Representative images of selected Cyterm-RFP fusions in HeLa cells 24 h after transfection. In reading order from left to right and from top to bottom the RFP in the Cyterm-RFP fusion was: mScarlet and mScarlet-I; mScarlet-I3-NCwt and mScarlet-I3, mCherry and TagRFP-T; and mScarlet3 and FusionRed

FIG. 15: mScarlet3 as fusion tag for cellular localization studies. In reading order from left to right and top to bottom mScarlet3 was fused to KDEL (endoplasmic reticulum) and alfa-tubulin (microtubule cytoskeleton); EB3 (+ends of microtubules) and Lamin (nuclear envelope); Giantin (Golgi apparatus) and 4xmito (mitochondria); LCK (plasma membrane) and life act (actin cytoskeleton); Peroxi (peroxisomes) and 3xNLS (nuclei) and expressed in HeLa cells.

FIG. 16: reduced cytotoxicity of mScarlet3 and mScarlet-I3 as compared to mEGFP and mScarlet (higher ratios are better). Small symbols refer to individual experiments, the large symbols indicate the mean ratio.

### SPECTROSCOPIC CHARACTERIZATION AND INTRINSIC BRIGHTNESS

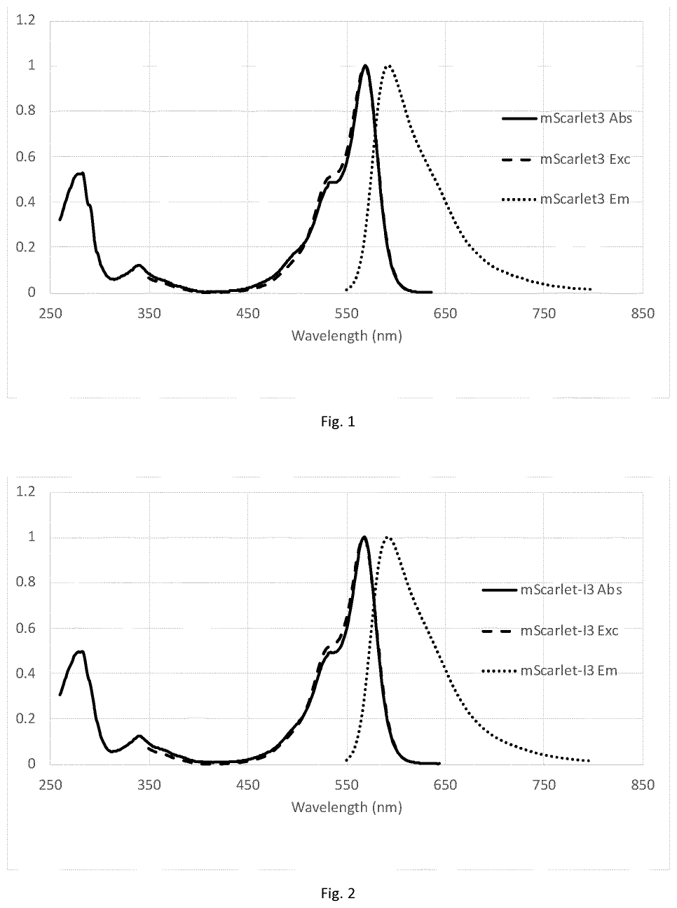

mScarlet3 (SEQ ID NO: 31) and mScarlet-I3 (SEQ ID NO: 35) have major improvements as compared to their ancestors mScarlet (SEQ ID NO: 1) and mScarlet-I (SEQ ID NO: 3), respectively. In Table I below the main bulk spectroscopic properties of these purified RFPs are indicated. The absorbance and fluorescence spectra (and maxima) are very similar of all 4 RFPs with absorbance maxima at 569 nm and emission maxima at 592-594 nm (see also FIGS. 1 and 2).

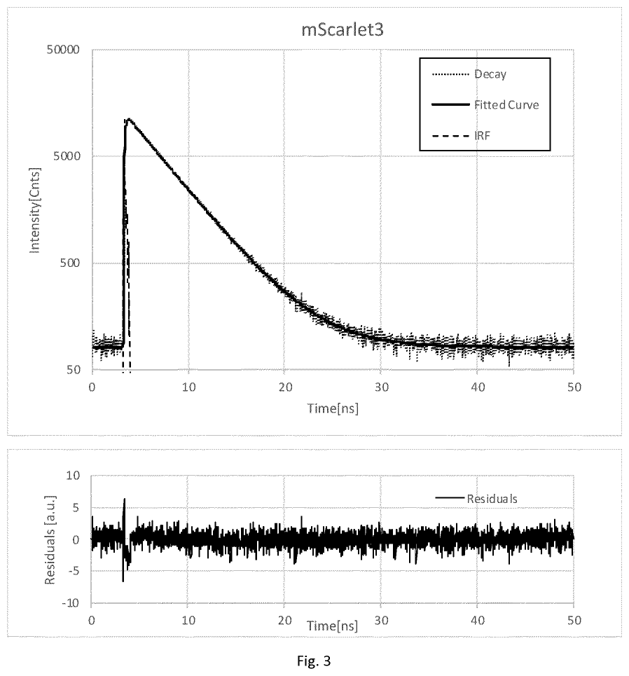

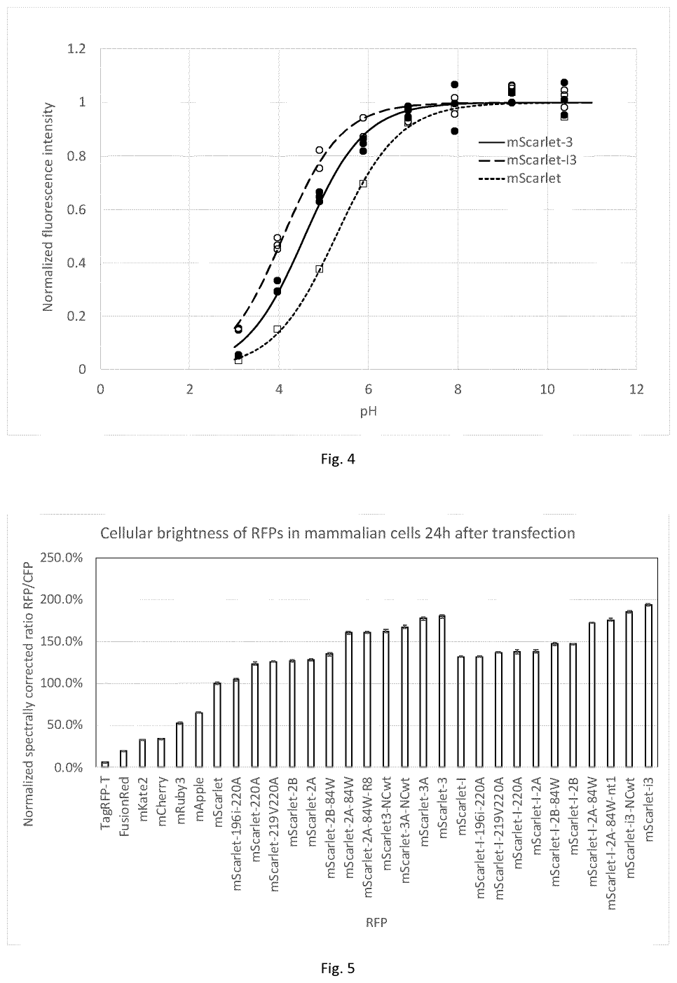

The extinction coefficients are very similar with values of 100000-105000 M⁻¹ cm⁻¹.Yet the quantum yield of mScarlet3 is 5% points improved as compared to mScarlet (Bindels et al., 2015). The 75.1% quantum yield is the highest quantum yield described to date for any monomeric RFP belonging to the same spectral class as mScarlet. The quantum yield for mScarlet-I3 is increased from 54% to 64.5% as compared to mScarlet-I. The increased quantum yields also contributed to higher fluorescence lifetimes (see FIG. 3 for mScarlet3). The 396 ns monoexponential decay time for mScarlet3 is the longest described to date of monomeric RFPs belonging to the Scarlet spectral class at physiological pH. The intrinsic brightness (the product of extinction coefficient and quantum yield) of mScarlet is also record high (78) for monomeric RRPs belonging to the mScarlet spectral class, a 10% increase of the previous record held by mScarlet (71). Finally, both mScarlet3 and mScarlet-i3 display a lower pK as compared to mScarlet and mScarlet-I. For instance, at pH 5 mScarlet3 and mScarlet-i3 retain more than 60% of their maximal fluorescence intensity, whereas at pH 5 mScarlet is already quenched to less than 40% of its maximal fluorescence intensity (see FIG. 4). As a consequence mScarlet3 and mScarlet-I3 are highly fluorescent also in acidic endomembrane compartments in cells and are hardly affected by pH changes near the normal physiological pH of 7.4, both contributing to an extended usefulness of mScarlet3 and mScarlet-I3 in cell-based fluorescence experiments. These data support the claim of superior in vitro spectroscopic properties and intrinsic brightness of mScarlet3 and mScarlet13.

TABLE I
spectroscopic properties of purified mScarlet RFP and its variants

| | Abs | Em | EC | | | | | n (Hill |
|---|---|---|---|---|---|---|---|---|
| | max | max | 1000 | | | | | coefficient |
| Protein | (nm) | (nm) | M⁻¹cm⁻¹ | QY (%) | Tau (ns) | EC*QY | pK | pK) |
| mScarlet | 569 | 593 | 100 ± 3 | 70.4 ± 0.8 | 3.86 ± 0.01 | 71 ± 2 | 5.3 ± 0.2 | 0.79 ± 0.22 |
| mScarlet-I | 569 | 594 | 104 ± 3 | 54 ± 4 | 3.01 ± 0.04 | 57 ± 4 | 5.4 ± 0.4 | 0.52 ± 0.19 |
| mScarlet-220A | 569 | 594 | 98 | 70 | n.a. | 69 | n.a. | n.a. |
| mScarlet-I-220A | 569 | 595 | 104 | 52 | n.a. | 54 | n.a. | n.a. |
| mScarlet-2A | 568 | 594 | 101 | 73 | 3.900 ± 0.003 | 74 | n.a. | n.a. |
| mScarlet-2A- | 569 | 594 | 99 | 74 | 3.960 ± 0.007 | 73 | n.a. | n.a. |
| 84W | | | | | | | | |
| mScarlet-2B | 569 | 595 | 95 | 68 | 3.793 ± 0.003 | 64 | n.a. | n.a. |
| mScarlet-2B- | 569 | 595 | 96 | 73 | 3.882 ± 0.007 | 70 | n.a. | n.a. |
| 84W | | | | | | | | |
| mScarlet-I-2A | 568 | 597 | 101 | 58 | 3.352 ± 0.006 | 55 | n.a. | n.a. |
| mScarlet-I-2A- | 568 | 595 | 103 | 64 | 3.553 ± 0.004 | 66 | n.a. | n.a. |
| 84W | | | | | | | | |
| mScarlet-I-2B | 569 | 597 | 102 | 54 | 3.47 ± 0.08 | 55 | n.a. | n.a. |
| mScarlet-I-2B- | 568 | 595 | 100 | 65 | 3.050 ± 0.008 | 65 | n.a. | n.a. |
| 84W | | | | | | | | |
| mScarlet-I3, | 568 | 592 | 102 ± 0.5 | 66.4 ± 0.7 | 3.634 ± 0.004 | 67 ± 1 | 4.1 ± 0.1 | 0.76 ± 0.16 |
| NCwt | | | | | | | | |
| mScarlet3 | 569 | 592 | 104 ± 1 | 75.1 ± 1 | 3.962 ± 0.003 | 78 ± 1 | 4.5 ± 0.1 | 0.70 + 0.04 |
| mScarlet-I3 | 568 | 592 | 105 ± 2 | 64.5 ± 1.1 | 3.638 ± 0.002 | 69 ± 2 | 4.1 ± 0.1 | 0.70 ± 0.04 |

### Cellular Brightness in Mammalian Cells

Intrinsic brightness only reports on the brightness of a fully maturated purified RFP, it does not report on the speed and extent of maturation, which greatly affect the observed cellular brightness of RFPs in cells. In FIG. 5, the cellular brightness of mScarlet3 and mScarlet-I3 in mammalian cells is compared to the main existing RFPs in the same spectral class. The cellular brightness was measured by co-expression of a cyan reference protein (mTurquoise2) at a 1:1 ratio with each RFP by transfection of a polycistronic vector carrying a P2A sequence, for method description see below and (Bindels et al., 2020). The red to cyan fluorescence ratio was measured for 200-500 individual cells and the mean RFP/CFP fluorescence ratio was determined, corrected for spectral throughput considering the excitation and emission bandpasses of the microscope and the ratios were subsequently normalized to mScarlet. mScarlet-I, despite its lower intrinsic brightness was the brightest RFP known to date in cells owing to its faster and more complete maturation in cells as compared to the other RFPs (see also below). Yet it is clear that both mScarlet3 and mScarlet-I3 substantially outperform both mScarlet and mScarlet-I and that they currently are by far the brightest (monomeric) RFPs in cells. The cellular brightness of mScarlet3 and mScarlet-I3 in cells is 76-79% increased as compared to mScarlet and 33-36% increased as compared to mScarlet-I. As compared to the relatively good maturating and still widely used RFP mCherry, the increase is >5-fold. These data support the claim that mScarlet3 and mScarlet-I3 are the brightest monomeric RFPs after expression in mammalian cells. In FIG. 5, also the intermediate variants found in the evolution from mScarlet to mScarlet3 and from mScarlet-I to mScarlet-I3 are indicated. Also, the immediate precursors of mScarlet3 and mScarlet-I3 with original N and C termini (referred to as mScarlet3-NCwt and mScarlet-I3-NCwt) are indicated in FIG. 5. This figure indicates that there is only a very modest influence of the new N and C-termini (Valbuena et al., 2019) on the final cellular brightness observed in cells, and hence that the other mutations cause the major increase in cellular brightness.

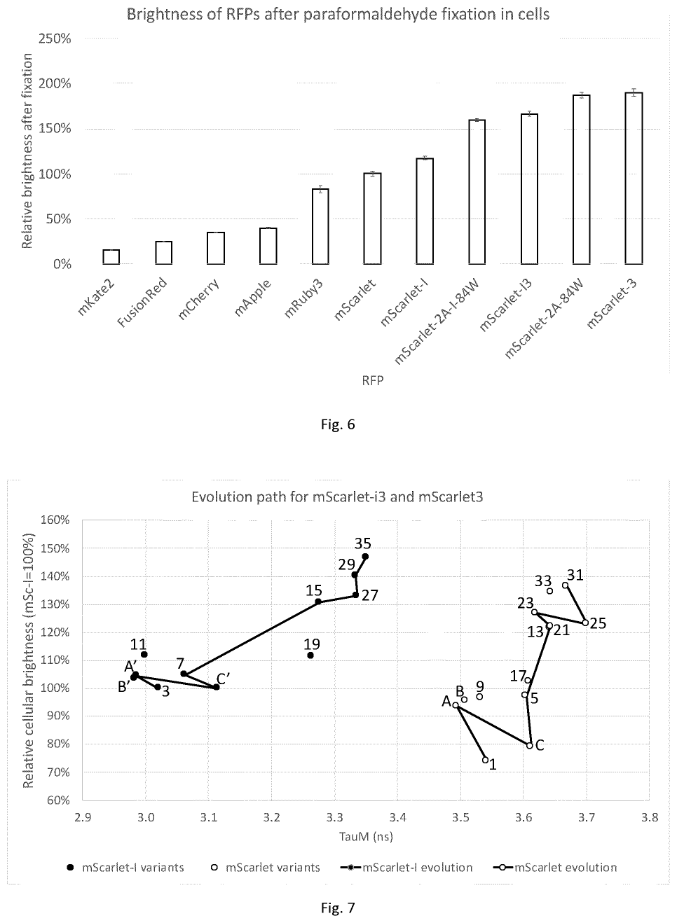

To check how the new Scarlet variants perform after chemical fixation, the cellular brightness of the new variants was also checked in cells after a 20 min fixation with 4% paraformaldehyde. In FIG. 6, it can be inferred that mScarlet3 and mScarlet-I3 are the brightest monomeric RFPs after fixation (90% and 66% brighter than mScarlet after fixation, respectively). It is also clear that mScarlet3 is much brighter after fixation than earlier developed RFPs such as mKate2, FusionRed, mCherry, mApple or mRuby3 (a difference of a factor 11.8, 7.6, 5.4, 4.8 and 2.3, respectively). This supports the claim that both mScarlet3 and mScarlet-I3 outperform other RFPs after chemical fixation. These experiments indicate that mScarlet3 and mScarlet-I3 are also suitable as red fluorescent markers compatible with immunohistochemistry and cytochemistry fixation protocols.

### Evolutionary Trajectory

By quantitative screening on multiple parameters simultaneously (Bindels et al., 2020), we could optimize both intrinsic brightness and maturation extent at the same time, which proved essential to not again end up with a compromise between these two important parameters. In FIG. 7, the diverse mScarlet variants that were isolated during evolution are indicated in a two-dimensional scatterplot. Variants with increased fluorescence lifetime and hence increased intrinsic brightness are found at the right side of the plot (i.e., all variants evolved from mScarlet have higher intrinsic brightness than those evolved from mScarlet-I (T74I)). Variants with increased cellular brightness in cells (product of intrinsic brightness and maturation extent) are found at the upper side of the plot.

From this figure it is clear that certain mutations can increase maturation but concomitantly decrease the intrinsic brightness, such as T74I (compare 1 with 3) and G220A (compare 3 with A′ and 1 with A). Other mutations such as V196I do the opposite: it increases intrinsic brightness at the cost of cellular brightness (compare A with C and A′ with C′). Interestingly, the Y84W mutation increases both parameters (compare 5 with 21 and 7 with 15). The internal mutations in mScarlet that all influence the balance between intrinsic and cellular brightness (T74I, Y84W, V196I, Y194F and G220A) all cluster around a single pocket in the 3D structure of mScarlet. These data support that all variants show increased cellular brightness compared to mScarlet (compare 1 with all the other points).

### Completeness of Maturation

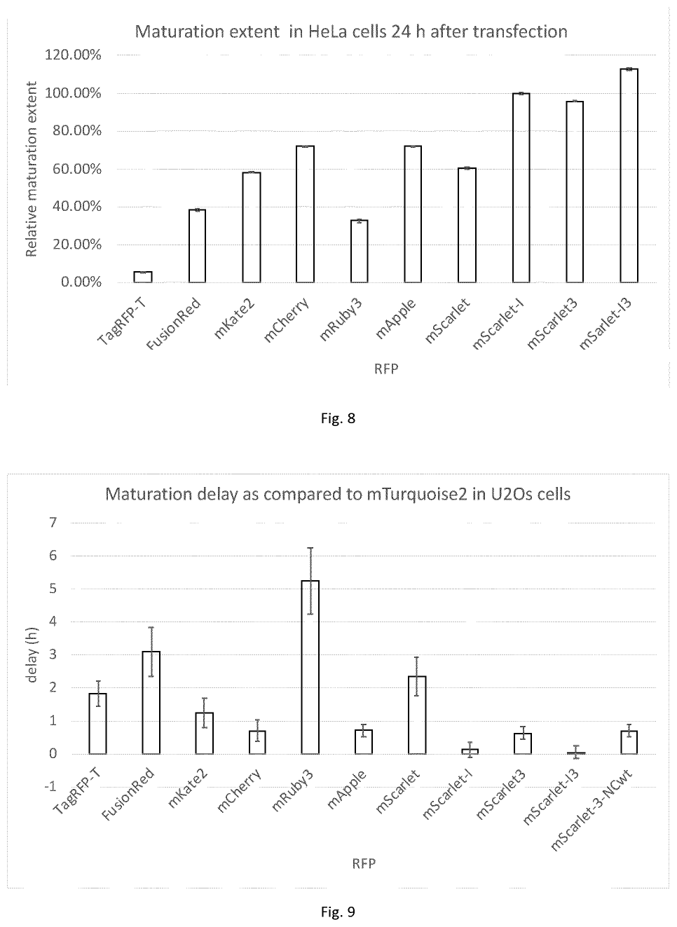

By comparing the intrinsic brightness (Table 1) and the cellular brightness observed in cells after 24 h (FIG. 1) we can infer the extent of maturation of these RFPs. The most complete maturating RFP known to date was mScarlet-I. It can be seen that after 24 h, mScarlet only maturates to 60% of the extent of mScarlet-I (FIG. 8). mScarlet3 maturates almost to the same extent as mScarlet-I, which is a huge improvement. The mScarlet-I3 is even more complete maturating than mScarlet-I (112%), making it the most complete maturating RFP known to date. These data support the claim that mScarlet3 and mScarlet-I3 are 100% complete maturating RFPs.

### Maturation Speed

Using the same expression vector as for the brightness experiments, we compared the delay of the development of red fluorescence and cyan fluorescence in mammalian cells. This experiment revealed maturation problems for several RFPs, for instance mRuby3, FusionRed and mScarlet have a maturation delays of 2.3-5.2 h as compared to the fast-maturating mTurquoise2 reference FP. It can be seen that mScarlet3 maturates 4× faster than mScarlet (which is a major improvement). The maturation delay of mScarlet3 of about half an hour is similar or even slightly faster as compared to the fast-maturating RFPs mCherry and mApple. Together with the increased intrinsic brightness this causes the 76% increase in cellular brightness in cells of mScarlet3 as compared to mScarlet. Owing to the T74I mutation present in mScarlet-I and mScarlet-I3, the maturation of these two latter RFPs is record fast and not significantly delayed as compared to mTurquoise2. The mScarlet3-NCwt variant with unchanged N and C terminus revealed no significant differences with mScarlet3 (not shown here). These data support the claim that mScarlet3 and mScarlet-I3 are rapidly maturating RFPs.

### Protein Stability

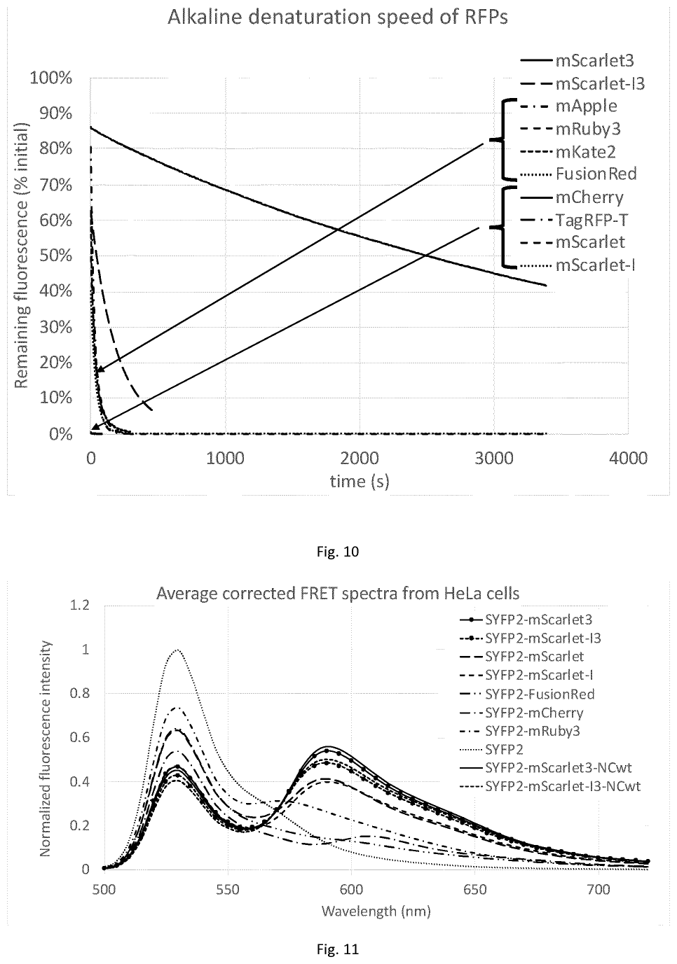

In the alkaline denaturation experiments required for the determination of the extinction coefficients (see Table 1), we found a surprising novel property of mScarlet3, which was a remarkable stability at highly alkaline pH. Where purified mScarlet and mScarlet-I denature instantly (<1s) upon elevation of the pH to 13.5, mScarlet3 and mScarlet-I3 withstand denaturation for prolonged time with half times of 45 and 2.5 minutes, respectively. By comparing RFP intermediates in the evolution of mScarlet to mScarlet3, we found a substantial stabilizing effect of the single mutation Y84W. We found similar kinetics for mScarlet-I3 and the mScarlet-I3-NCwt variant with identical N and C termini as mScarlet-I (not shown), indicating that the enhanced protein stability does not depend on the new N and C termini. Therefore, we conclude here that the internal mutations Y84W, Y194F, V196I, G220A and the supporting external mutations have a large stabilizing effect in the protein structure. They greatly reduce alkaline denaturation speed (FIG. 10), they cause more complete (FIG. 8) and faster maturation (FIG. 9) and they stabilize the resulting RFP and thereby increase the fluorescence quantum yield and fluorescence lifetime (Table 1), together resulting in the remarkable cellular brightness increase in mammalian cells (FIG. 5). These data support the claim that mScarlet3 and mScarlet-I3 have enhanced protein stability and are resistant against extreme alkaline conditions.

### FRET Sensing

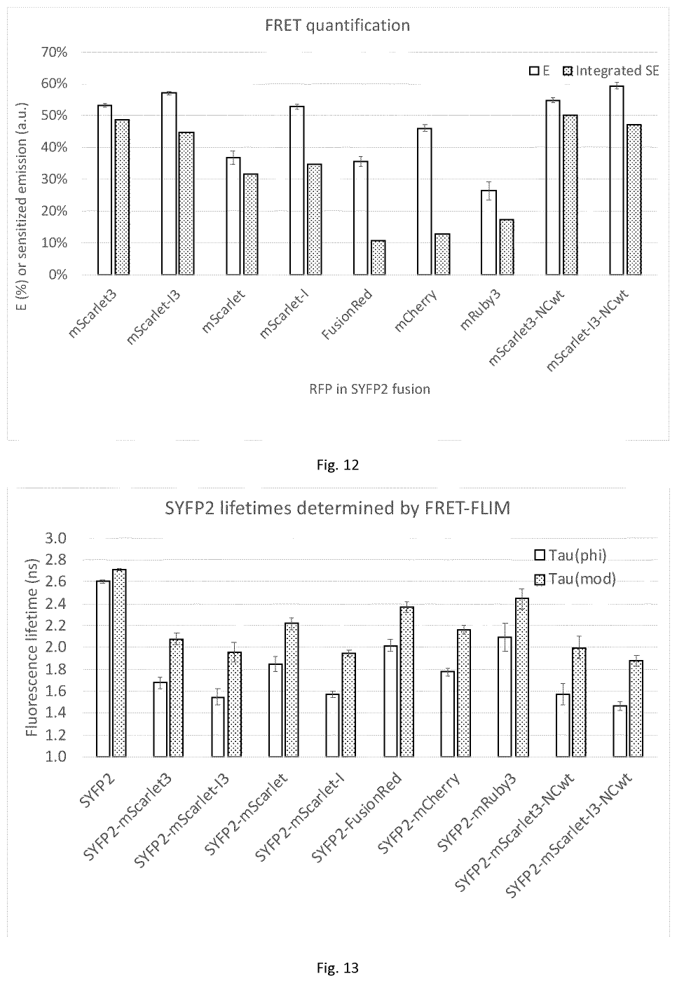

Considering the enhanced intrinsic brightness and maturation in cells it was expected that mScarlet3 and mScarlet-I3 are superior FRET acceptors. To this end direct fusions were generated with SYFP2 (a bright monomeric yellow fluorescent protein) and several monomeric RFPs and the extent of FRET was measured with SPIM and FLIM. In FIG. 11 the average corrected spectra are shown that were obtained from HeLa cells 48 after transfection upon excitation at 500 nm. The extent of FRET can be easily inferred from the ability of the diverse RFPs to quench the SYFP2 donor (500-550 nm). This quenching is mainly determined by the spectral overlap between the fluorescence emission spectrum of SYFP2 with the absorbance spectrum of the different RFPs (not shown) and the maturation efficiencies of the RFPs. It can be clearly seen that the best quenching of SYFP2 is done by mScarlet-I3 (57%) followed by mScarlet3 and mScarlet-I (both 53%), mCherry (45%), mScarlet and FusionRed (both 36%), and mRuby3 (26%), see also FIG. 12. This order is nicely in line with the extent of maturation of these RFPs as shown in FIG. 8, and it confirms that the extent of maturation of mScarlet3 is equal to mScarlet-I, and that mScarlet-I3 has even slightly better maturation. At higher wavelengths (>570 nm), the sensitized emission due to FRET and direct excitation of the acceptor contribute to the detected fluorescence signal, which is strongly dependent on the quantum yield and extent of maturation of the respective RFPs. Here it is seen that despite the slightly lower YFP quenching (and hence FRET efficiency), mScarlet3 produces more red emission owing to its higher intrinsic brightness than mScarlet-I3.

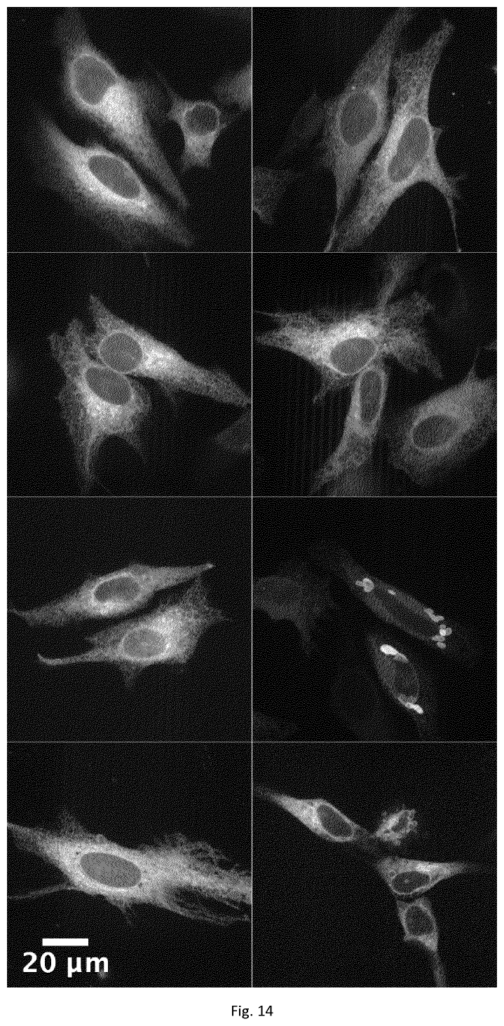

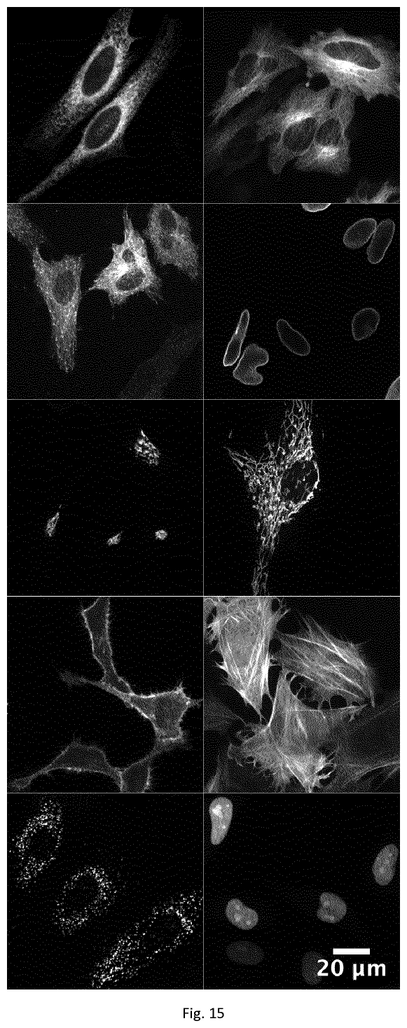

The same cells were analyzed with frequency-domain FRET-FLIM, which is a technique that can quantify the donor quenching by measuring the donor fluorescence lifetime independent of red fluorescence emission and protein expression levels. In case of FRET the SYFP2 lifetimes are reduced. In FIG. 13, results are shown of the average lifetime observed in >20 cells in at least 4 independent FLIM acquisitions in HeLa cells expressing either unfused unquenched SYFP2 or several SYFP-RFP fusion proteins 48 h after transfection. It can be easily inferred that mScarlet3 and mScarlet-I3 efficiently lower the SYFP2 lifetime from 2.60/2.71 ns to 1.68/2.07 ns and 1.55/1.96 ns respectively, which is lower as compared to the ancestor mScarlet (1.85/2.22 ns) and similar as compared to mScarlet-I (1.57/1.95) ns (tau(phi)/tau(mod)). From the phase lifetime, the FRET efficiencies of the diverse RFP fusions are 41% for mScarlet-I3, 40% for mScarlet-I, 36% for mScarlet3, 32% for mCherry, 29% for mScarlet, 22% for FusionRed and 20% for mRuby3. Hereby FRET-FLIM shows that both mScarlet3 and mScarlet-I3 are much better quenchers of SYFP2 than other monomeric RFPs such as FusionRed, mCherry and mRuby3. Together, these data show that mScarlet3 and mScarlet-I3 are the best FRET acceptors currently available among the monomeric RFPs and that they both outperform mScarlet-I in spectral-FRET. Given these properties, this makes these RFPs the preferred RFPs for building FRET sensors, which is expected to be a major future application. These data support the claim that mScarlet3 and mScarlet-I3 are superior FRET acceptors and preferred RFPs for building red-shifted FRET sensors.

### Oligomeric State

To make sure that the enhanced mScarlet variants remain monomeric, we performed an OSER experiment (Costantini et al., 2012). In the OSER experiments, diverse RFPs were fused to the CytERm ER-localizing sequence that encodes an integral ER-membrane protein fused to RFP with RFP facing the cytoplasm. A tendency of the RFP to dimerize causes the aggregation of smooth endoplasmic reticulum in so called whorl structures or OSERs. These can be visualized with fluorescence microscopy and counted. The result is shown in Table II and representative cells are shown in FIG. 14. It can be inferred that mScarlet3 and mScarlet-I3 hardly induce OSER structures and that the cells expressing CytERm-mScarlet3 or CytERm-mScarlet-I3 display a regular ER labeling, in contrast to for instance Cyterm-TagRFP-T.

TABLE II
evaluation of oligomeric structure of RFPs

| | % cells with | Number of cells |
|---|---|---|
| Cyterm fusion: | OSER | analyzed |
| mScarlet3 | 7% | 139 |
| mScarlet-I3 | 5% | 39 |
| mScarlet | 8% | 39 |
| mScarlet-I | 8% | 80 |
| mCherry | 18% | 87 |
| TagRFP-T | 64% | 132 |
| dTomato | 82% | 114 |
| mEGFP | 11% | 62 |

Performance of mScarlet3 As Fusion Tag for Subcellular Localization Studies

One of the main applications of fluorescent proteins is the usage as fusion tag in cellular studies. We made several fusion constructs of mScarlet3 and expressed these in HeLa cells (see FIG. 15). The nice microtubule cytoskeleton labelling upon expression of alfa-tubulin-mScarlet3 as observed in the top left image of FIG. 15 underlines the monomeric behavior and excellence performance of mScarlet3 as fusion tag. All fusion constructs clearly provided very clear organelle or cytoskeletal structure labelling from which we conclude that mScarlet3 is an excellent fusion tag for cellular protein localization studies.

mScarlet3 and mScarlet-I3 have Reduced Cytotoxicity

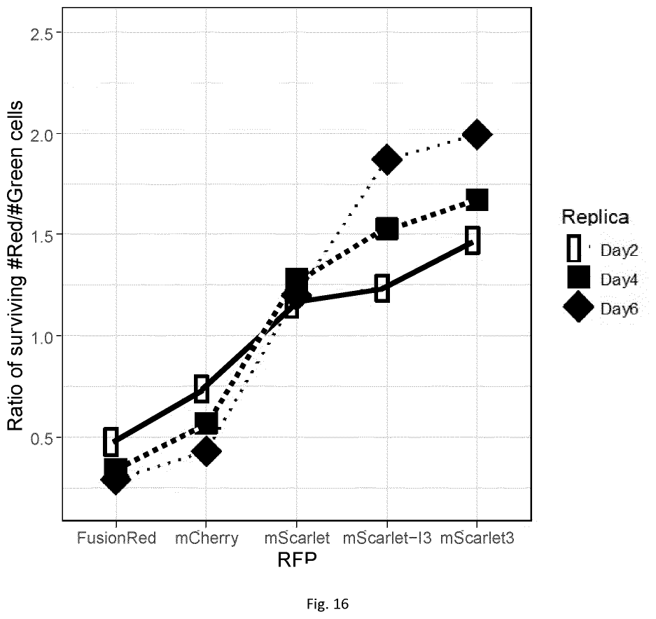

To evaluate the toxicity of overexpression of mScarlet3 and mScarlet-I3, a cytotoxicity experiment was performed by separate transfection of identical amounts of plasmids encoding cytosolic mEGFP (control), mScarlet, mScarlet3, mScarlet-I3, mCherry or mFusionRed. After two days all cells were split and the control mEGFP cells were mixed with each of the RFP transfected cells. The 5 resulting cell mixtures were subsequently grown and split two additional times with an interval of two days. For each individual mixture 4-5 biological replicates were split/grown and during the split two aliquots for each biological replicate was analyzed for the number of green and red fluorescent cells by fluorescence microscopy, amounting to 8-10 red/green cell number ratios per time point per RFP. In FIG. 16, these ratios are plotted for each mixture at indicated times after transfection and this clearly shows that for mScarlet3 and mScarlet-I3, the ratio of surviving red cells over green cells increased from day 2 to day 6 after transfection, indicating reduced cytotoxicity. (Valbuena et al., 2019) showed that the new N and C termini result in reduced cytotoxicity of mScarlet-I in bacteria, but they could not determine a beneficial effect in mammalian cells. Therefore, it is possible that not the new N and C termini but all other mutated amino acids found in mScarlet3 and mScarlet-I3 contribute to the decreased cytotoxicity. These data show that both mScarlet3 and mScarlet-I3 are ideally suited for prolonged live cell experiments due to reduced cytotoxicity.

### Methods

### General Methods

All red fluorescent proteins (RFPs) were cloned into a pDRESS plasmid (Addgene 130509) (Bindels et al., 2020) or pDX vector (a modified TriEX vector, expressing a FP in both bacteria, under a rhamnose promoter, and in mammalian cells, under a CMV promoter) using the AgeI and BsrGI restriction sites. mRuby3 (Bajar et al., 2016), TagRFP-T (Shaner et al., 2008), mApple (Shaner et al., 2008), mCherry (Shaner et al., 2004) mKate2 (Shcherbo et al., 2009) and FusionRed (Shemiakina et al., 2012) were obtained as described in (Bindels et al., 2016). Mammalian cell imaging was done with U2OS cells (HTB-96, ATCC) or HeLa cells (CCL-2 ATCC). Mammalian cells were grown in 24 well plates with glass bottom (MatTek P24G-1.5-13-F) in DMEM (61/965,059, Thermo Fisher Scientific) containing 10% fetal bovine serum (10270106, Thermo Fisher Scientific) or with colorless DMEM 1159446 Thermo Fisher Scientific) supplemented with 1% of Glutamax (11574466 Thermo Fisher Scientific) under 7% humidified CO2 atmosphere at 37° C. For transfection polyethylenimine (PEI) in ddH2O (1 mg/ml, pH 7.3, 23966, Polysciences) was used. The transfection mixture was prepared in Opti-MEM (31985047, Thermo Fisher Scientific) with 2 μl of 2 g/l PEI solution and 50-200 ng plasmid. For some transfections, carrier DNA (empty plasmid) was added to prevent overexpression. The transfection mixture was incubated for 20-45 min. Cells were used for imaging 15-48 h after transfection.

### Mutagenesis

mScarlet variants were obtained by site-directed and random mutagenesis using standard protocols, see (Bindels et al., 2016). Evolution started from mScarlet by introducing G220A, followed by V196I targeted mutagenesis. With random mutagenesis the combination of T107S, G156V and E219V was found to be beneficial. A new template mScarlet-2A (Seq-ID 5,6) was ordered as geneblock carrying T107S, G156V, V196I, E219V and G220A as mutations relative to mScarlet. Considering the quantum yield increase found in mutating F84Y (final step in the evolution of mScarlet (Bindels et al., 2016)), an even bulkier mutation Y84W was introduced in mScarlet-2A by targeted mutagenesis, yielding mScarlet-2A-84W (=mScarlet+Y84W, T107S, G156V, V196I, E219V and G220A) (Seq-ID 13,14). We also introduced T74I (this is the mutation that generated mScarlet-I from mScarlet) into mScarlet-2A-84W, which was dubbed mScarlet-I-2A-84W (Seq-ID 15,16). These two new templates were subjected to an additional round of random mutagenesis. From screening of several variants with increased maturation, a variant with enhanced cellular brightness was found: mScarlet-2A-84W-R8 (Seq-ID 21,22) with two mutations T109A, K183R. From analysis of other bright variants, two subsequent mutations were introduced with targeted mutagenesis: first Y194F (Seq-ID 23,24) and then K48R yielding mScarlet3-NCwt (Seq-ID 25,26). With 2CR new N and C termini (Valbuena et al., 2019) were introduced (Δ1, V2M,S3D,K4S, G5T and M227S, D228G,E229G, S230S, A231,A232) yielding mScarlet3 (Seq-ID 31,32). In the random mutagenesis of mScarlet-2A-1-84W a combination of 6 mutations originating from different mutated variants were found to be beneficial. These mutations were introduced by ordering a new template mScarlet-I-2A-84W-nt1 (Seq-ID 27,28) containing K93R, N99I, A105T, T128G, K140R, Y194F relative to the mScarlet-2A-1-84W template. Subsequent scrutinizing demonstrated that reversal of R93K was beneficial yielding mScarlet-I3-NCwt (Seq-ID 29,30), With PCR new N and C termini were introduced (Δ1, V2M, S3D,K4S, G5T and M227S, D228G,E229G, S230S, Δ231, Δ232) yielding mScarlet-I3 (Seq-ID, 35,36).

### Protein Purification

His-tagged recombinant RFPs were purified from E coli bacteria as described (Bindels et al., 2016) except for the final affinity purification and dialysis steps. For affinity purification the crude E coli protein extract was obtained from defrosted E coli pellets and incubation on ice with 5 ml ST buffer (20 mM Tris/HCl, 200 mM NaCl, pH 8.0) supplemented with lysozyme (1 mg/ml, L7651, Sigma-Aldrich), benzoase nuclease (5 unit/ml, Merck/Millipore, 71205-3) and 50 ul 100× Halt Protease Inhibitor Cocktail (Thermo Scientific 87785). After 2 h of incubation, 100 μl of 10% (v/v) NP-40 (Thermo Fisher Scientific, 85124) was added to the lysate after which it was centrifuged (30 min, 40,000 g, 4° C.). The supernatant was added to 2 ml of Co²⁺ loaded HisPurTM Cobalt Superflow Agarose resin (Thermo Scientific, 25228) and incubated for at least 1 hour at 4° C. The resin was washed with 9-10 times with 2 ml wash buffer (ST-buffer supplemented with 15 mM imidazole) until no detectable protein (as measured by OD 280) was washed from the resin. His-tagged proteins were eluted with 2×1 ml elution buffer (ST buffer supplemented with 150 mM imidazole). The eluent was desalted and obtained in a 10 mM Tris-HCl pH 8.0 solution using Sephadex-G25 desalting columns (GE Healthcare 17-0852-01). Proteins were short-term stored at 4° C., or flash frozen and stored at −80° C. for long-term storage.

### Spectroscopy

### Extinction Coefficient

Purified proteins were diluted in PBS (50 mM Na₂HPO₄—NaH₂PO₁, 137 mM NaCl, 2.7 mM KCl, pH 74). Absorbance spectra were acquired with a spectrophotometer (Libra S70, Biochrom). The spectra were recorded in the wavelength range of 260-700 nm, with a step size of 1 nm. PBS was used as a background reference. The samples were diluted such that the absorbance of the red chromophore peaked between 0.15 and 0.5. To denature the RFPs 10-200 μl 10 M NaOH was subsequently added to the samples, which was directly mixed by pipetting. Spectra were acquired continuously after addition of the sodium hydroxide until the absorbance spectra showed a complete loss of the absorbance peak associated with the red chromophore and displayed only the peak associated with the green chromophore at 457 nm. This absorbance spectrum was used for further analysis, and if necessary, the average absorbance value in the wavelength range 670-680 nm was subtracted from the spectra, in order to correct fora minor offset. The concentration of the denatured green chromophore was calculated assuming an extinction coefficient of 44,000 M⁻¹ cm⁻¹ at 457 nm for the green chromophore in the denatured RFP (Gross et al., 2000)(Shagin et al., 2004). Based on the concentration of the red chromophore the extinction coefficient for the red chromophore was determined at the maximum absorbance wavelength. The above procedure was repeated at least three times per RFP variant and the average extinction coefficient was calculated.

### Quantum Yield

Purified proteins were diluted in PBS (50 mM Na₂HPO₁—NaH₂PO₄, 137 mM NaCl, 2.7 mM KCl, pH 7.4). Absorbance spectra were recorded with a spectrophotometer (Libra S70, Biochrom) in the wavelength range 260-700 nm with a step size of 1 nm. PBS was used as a background reference. Three dilutions of each RFP variant were prepared with an absorbance at 540 nm (A₅₄₀) of 0.005<A₅₄₀<0.05. Fluorescence emission spectra were taken from the same sample cuvette with a fluorimeter (Model FP-8500, Jasco with a red extended PMT tube model R928-23). The excitation wavelength was set at 540 nm, the emission spectrum was recorded from 550 to 800 nm with a step size of 1 nm at a scan speed of 200 nm·min⁻¹. The excitation as well as the emission slits were set at 5 nm. To obtain more accurate A₅₄₀absorbance values of the low absorbing samples, their absorbance spectra were fitted to an absorbance spectrum of the same RFP at high concentration (OD 0.1-0.3) and a variable (constant) offset value using a linear least squares fit. The A₅₄₀ absorbance of the quantum yield samples was obtained from the fitted spectral component. Fluorescence spectra were background corrected by subtracting a spectrum measured with PBS. The emission spectra were corrected for spectral sensitivity using a calibrated white light source (ESC-842, Jasco) and the spectral area (1_{cm}) was obtained by integrating from 550-800 nm. The absorbance at 540 nm (A₄) was plotted versus the area under the emission spectrum, subsequently the slope of the line was determined using linear regression. The regression lines were constrained to go through the origin, hence I_{cm}=α·A₅₄₀.

Equation 1 was used to calculate the quantum yield:

$$Q {Y}_{s} = {QY}_{r} \frac{{a}_{s}}{{a}_{r}} \qquad Eq . 1$$

In Equation 1 QY denotes the quantum yield (s and r denote sample and reference RFP, respectively) and a corresponds to the acquired slope. mScarlet was used as a reference with a quantum yield of 0.704 (Bindels et al., 2016).

### Fluorescence Lifetime

Fluorescence lifetime measurements of purified RFPs diluted in PBS (50 mM Na₂HPO₄—NaH₂PO₄, 137 mM NaCl, 2.7 mM KCl, pH 7,4) were performed at an Olympus FV1000 confocal microscope equipped with a PicoHarp 300 TCSPC module (PicoQuant, Germany) as described (Bindels et al., 2016).

### pH Dependence of RFP Fluorescence Intensity

A pH buffer series was created (pH 3-10) using a universal 50 mM citric acid, 50 mM phosphoric acid and 50 mM boric acid, 100 mM NaCl buffer. Buffers at the desired pH were made by titrating a 2-times concentrated stock solution with 1M NaOH (Merck 109137) and adjusting the volume obtain a two-fold dilution. Citric acid and phosphoric acid (85%) were from Merck (art 818707 and 563, respectively), boric acid was from Sigma (B-0252). The final pH value was measured 24 h after preparation at room temperature and yielded pH values of 2.96, 3.90, 4,85, 5.94, 6.89, 8.16, 9.21 and 10.75. Fluorescence emission and absorbance Purified RFPs (section protein purification) were diluted in a black μ-clear 96 wells plate (655090, Greiner). For each pH triplicate samples for one RFP were made ranging from pH 3.0 to 10.8. With a Biotek FL-600 fluorescence plate reader the fluorescence intensity was measured using a 555/25 excitation filter and a 620/40 emission filter. The curves of pH versus the fluorescence of the samples, F(pH), was fitted using the Hill-function, Equation 2 to obtain the apparent pKa of the RFP and the Hill-coefficient n:

$$F ( pH ) = \frac{{F}_{max}}{1 + 1 {0}^{n ( pKa - p H )}} \qquad Eq . 2$$

### Stability at pH 13.5

Fluorescence intensity was monitored as a function of time in a fluorimeter (Model FP-8500, Jasco with a red extended PMT tube model R928-23) using 560 nm excitation and 610 nm emission (slits at 5 nm). Measurements were started with blanc PBS solution (50 mM Na₂HPO₄—NaH₂PO₄: 137 mM NaCl, 2.7 mM KCl, pH 7.4) to which 10 μl of purified RFP was added. After 1 min 100 μl of 2 M NaOH was added and the mixture was quickly mixed by pipetting up and down in the cuvette. To determine the stability of RFP fluorescence at alkaline pH, the blanc intensity was subtracted and the signal was normalized to the blanc-corrected RFP fluorescence signal at normal pH. The signal after addition of NaOH was corrected for dilution. After NaOH addition the final unbuffered NaOH concentration was 0.33 M, yielding a pH of 13.5.

### Cellular Microscopy-Based Methods

### Brightness Mammalian Cells

The cellular brightness of the diverse RFPs was determined in U20s and HeLa cells as described in (Bindels et al., 2020). Briefly, 50 ng of pDress vector encoding a 1:1 expression of mTurquoise2 cyan fluorescent protein and the respective RFP was mixed with 150 ng carrier DNA and transfected in mammalian cells in glass bottomed 24 well plates as described under general methods. 24 h after transfection cyan, yellow, and red fluorescence images were recorded on a Nikon wide field microscope. The widefield microscope consisted of an Eclipse Ti-E (Nikon) equipped with 440, 508 and 555 nm LEDs (SpectraX, Lumencor). The excitation light from these LEDs was passed through a 440/20, 510/24 or 550/15 nm bandpassses, respectively. For 440 nm and 508 nm excitation, a triple-band cube (MXU74157, Nikon) was used, for 555 nm excitation a quad band cube (MXU 71640, Nikon) was used. Emission was additionally filtered with a 479140, 550/49 nm or 593/46 nm bandpass (all from Semrock) placed in an optical filter changer (Lambda 10-B, Sutter instrument). For the three detected channels cyan, yellow, and red, the effective excitation and emission bands were cyan: 430-450 nm excitation, 459-490 nm emission, yellow: 498-523 nm excitation, 526-555 nm emission, red: 543-558 nm excitation and 570-616 nm emission. A 10×CFI Plan Apochromat NA 0.45 (Nikon MRD00105) objective was used. Images were acquired on an ORCA-Flash4.0V2 Digital CMOS camera (C11440-22CU, Hamamatsu Photonics). For each well a 5×5 tile of Images of 512×511 pixels each was acquired using a central ROI of 1024×1022 pixels with 2×2 binning and automated image stitching with 10% overlap, resulting in a final image size of 2253×2253 pixels corresponding to a 2.91×2.91 mm imaged area in each well. LED power was 10%, 20% and 10% for 440, 508 and 555 nm LEDs for the cyan, yellow and red images, respectively. Integration time per image was 60 ms, 200 ms and 60 ms for the cyan, yellow, and red images, respectively. The ratio of red to cyan fluorescence was calculated using the ratio_96-wells_macro_v7 as described (Bindels et al., 2020). The cellular brightness in mammalian cells was obtained by correcting these ratios for spectral throughput of the imaging device. To this end, spectra of the corresponding purified RFPs were used and the average excitation in the 543-558 nm excitation bandpass was calculated from the normalized excitation spectrum and the integrated fluorescence in the 570-616 bandpass was divided by the total integrated emission (550-800 nm). These corrections were normalized to the throughput for mScarlet. Consequently, the detected Red/Cyan ratios were multiplied by 1.0 for mScarlet, 1.015 for mScarlet-I, 0.985 for mScarlet3, 0,964 for mScarlet-13, 1.522 for mCherry, 0.919 for mApple, 0.667 for TagRFP-T, 0.696 for mRuby3, 1.245 for FusionRed and 3.066 for mKate2 to obtain the relative cellular brightness values corrected for spectral throughput.

Brightness in Mammalian Cells after Fixation.

The cellular brightness of the diverse RFPs following was also determined after paraformaldehyde fixation in HeLa cells. Transfection, imaging, and quantification was performed as described above for living cells, but prior to determining the red to cyan fluorescence ratio, cells were washed once with PBS and subsequently fixed for 20 minutes at room temperature with freshly prepared 4% paraformaldehyde in PBS. After fixation, cells were washed once with PBS and taken up in microscopy medium (140 mM NaCl, 5 nM KCl, 1 mM MgCl2, 1 mM CaCl2, 10 mM glucose, and 20 mM HEPES pH 7.4) and imaged on the same day.

### Maturation

The extent of maturation was calculated by dividing the corrected cellular brightness by the intrinsic brightness (see table 1) for the respective RFP and normalizing to mScarlet-1.

For measuring the maturation speed, the same cells, transfection and setup as described for the cellular brightness in mammalian cells was used. For image acquisition the yellow image was omitted and cyan and red single image using the full sensor ROI at 2×2 binning was used. Starting 5 hours after transfection, for each well in the 24 well plate a red and cyan image was recorded every 15 minutes for 24 h. ROIs were manually drawn around 6-12 individual cells in each timelapse that remained viable for prolonged time and showed steady FP accumulation overtime, but that started with no visible FP accumulation. The background corrected average intensity in the ROI was calculated for both the cyan and red channels. Subsequently, only traces that first showed a nonlinear increasing accumulation of both RFP and CFP fluorescence, followed by a linear increasing accumulation and ending with a slowing down of the increase were selected. Cells with discontinuous time traces (due to cell division or apoptosis) were discarded. The RFP and CFP trace were normalized to maximal intensity and a straight line was fitted to the curve at the point a maximal slope of normalized fluorescence increase with time was calculated. The intercept of this line with the time axis was calculated and the delay between the intercept found for the red and cyan curves were calculated for 6-15 individual cell traces and averaged.

### FRET-FLIM

For all RFPs, direct fusion constructs with SYFP2 were generated using PCR- and restriction enzyme-based cloning. To enable a good comparison, the linker between the SYFP and RFP was chosen such that a constant number of amino acids separated the two beta barrels in the fusion construct (i.e. correcting for differences in the length of N- and C-termini). Below in Table Ill, the linker sequence between the different RFPs and SYFP2 is described for the constructs used for the FRET studies depicted in FIG. 11-13.

TABLE III
Linker sequence of RFP-SYFP2 FRET constructs

| | | SEQ |
|---|---|---|
| RFP | | ID |
| fusion | Linker sequence | NO |
| mScarlet3- | QYERSVARHSTGGSGGSLYKVSKGEELFTGVVP | 37 |
| SYFP2 | | |
| mScarlet- | QYERSVARHSTGGSGGSLYKVSKGEELFTGVVP | 38 |
| I3-SYFP2 | | |
| mScarlet- | QYERSEGRHSTGGMDELYKMVSKGEELFTGVVP | 39 |
| SYFP2 | | |
| mScarlet- | QYERSEGRHSTGGMDELYKMVSKGEELFTGVVP | 40 |
| I-SYFP2 | | |
| FusionRed- | QHEVAVARYSTGGAGDLYKMVSKGEELFTGVVP | 41 |
| SYFP2 | | |
| mCherry- | QYERAEGRHSTGGMDELYKMVSKGEELFTGVVP | 42 |
| SYFP2 | | |
| mRuby3- | QREVAVAKYSNLGMDELYKMVSKGEELFTGVVP | 43 |
| SYFP2 | | |

Dark-shaded outline depicts (truncated) C-terminus of RFP, light-shaded outline depicts (truncated) N-terminus of SYFP2.

50 ng of the SYFP2-RFP fusion constructs, SYFP2 or RFP single FP constructs, together with 150 ng of carrier DNA were transfected in HeLa cells growing in glass-bottomed 24-well plates as described above. 46 h after transfection, the samples were analyzed with FRET-FLIM with a frequency-domain widefield FLIM setup as described (Bindels et al., 2020). Briefly, the setup consisted of a Nikon TiE inverted fluorescence microscope, with a Proscan-III automated stage, excitation filter wheel and a Lambert Instruments FLIM Attachment (LIFA) system including a Multi-LED light source and a LI2CAM detector (Lambert Instruments). For measuring the SYFP2 lifetime a 506 nm LED modulated at 40 MHz was used (Lambert Instruments), and the excitation light was additionally filtered with a 500/24-nm excitation filter (BrightLine single-band bandpass filter; Semrock, cat. no. FF01-500/24). A filter cube with a 523-nm dichroic mirror (Semrock, cat. no. Di02-R514), and a 542/27-nm emission filter (BrightLine single-band bandpass filter; Semrock, cat. no. FF01-542/27-25) was used to separate excitation from the SYFP2 fluorescence emission. A 40×CFI Plan Apochromat NA 0.95 air objective (Nikon) was used. For determining the phase and modulation of the modulated excitation light at the sample position, a diluted (OD<0.05) Alexa488 (Thermo Fisher Scientific) solution in PBS was used, for which a monoexponential fluorescence lifetime of 4.03 ns was assumed. FLIM measurements were done in culture medium at 37° C. under 5% CO₂ atmosphere. For each SYFP-RFP sample 3-4 FLIM stacks were acquired with a different field of view of approximately 20 cells each. The mean lifetime of all cells in each FLIM stack was determined and averaged for all stacks acquired from the same sample.

### FRET-SPIM

48 h after transfection, the medium of the same samples as described under FRET-FLIM was replaced with microscopy medium (140 mM NaCl, 5 mM KCl, 1 mM MgCl2, 1 mM CaCl2, 10 mM glucose, and 20 mM HEPES pH 7.4) and spectral images were acquired at room temperature using a home-built spectral imaging setup as described (Bindels et al., 2016; Vermeer et al., 2004). Briefly the setup consisted of a Zeiss Axiovert 200M fluorescence microscope, equipped with an HBO 100 Mercury lamp for excitation and an imaging spectrograph (Imspector V7, Specim, Finland) coupled to a CCD camera (ORCA ER, Hamamatsu, Japan). A 10× plan Neofluar NA 0.3 objective was used. Two spectral images were recorded: one with a 500/20 nm excitation filter (Chroma Technology Incorporation), a 80/20 dichroic mirror (20/80 bs, Chroma Technology Incorporation) and a LP530 nm emission filter (#46-059, Edmund optics worldwide) to record the full spectra and one with a 577/20 nm excitation filter, (D577/20, Chroma Technology Incorporation), a 596 nm dichroic mirror (600dcxr, Chroma Technology Incorporation), and a 630/60 nm emission filter (HQ630/60, Chroma Technology Incorporation) to record the direct excited RFP acceptor signal. The spectra were extracted from the spectral images and analyzed as described (Bindels et al., 2016). Briefly, the direct excited acceptor spectrum was subtracted by analyzing the spectra from single RFP transfected cells, and the remaining donor and sensitized emission acceptor signals were obtained by spectral unmixing with linear least squares method using the unfused SYFP2 donor spectrum and unfused RFP acceptor spectrum as reference. Typically, 10-30 single cell spectra were analyzed per SYFP-RFP fusion. The averaged spectra corrected for detector sensitivity and normalized to the unquenched donor signal were calculated.

### Localization

The Giantin DNA coding sequence of FRB-ECFP(W66A)-Giantin (67903, Addgene) was cloned in to pmScarlet3-C1, using the restriction enzymes BsrGI and BamHI. The following constructs pLifeAct-mTurquoise2 (36201, Addgene), pmTurquoise2-Mito (36208, Addgene), pmTurquoise2-H2A (36207, Addgene), pmTurquoise2-alfaTubulin (36202, Addgene), pmTurquoise2-Peroxi (36203, Addgene), EB3-mTurquoise2 (98825, Addgene), LCK-mTurquoise2 (98822, Addgene) and ER-mTurquoise2 (36204, Addgene) were digested with AgeI and BsrGI to exchange mTurquoise2 for mScarlet3. 4xmts-mScarlet3 and 3xnls-mScarlet3 were created by digesting 4xmito-mNeongreen (Addgaene 98875) and 3xnls-mNeongreen (Addgene 98875) with AgeI and BsrGI to exchange mNeongreen for mScarlet3. LaminB-mTurquoise2 (99830, Addgene) was digested with AgeI and BgIII to exchange mTurquoise2 for mScarlet3.

HeLa cells (CCL-2, ATCC) were seeded in uncoated glass-bottomed 24 well plates (MatTek P24G-1.5-13-F) and transfected with 50 ng plasmid, 150 ng carrier DNA and 2 μg PEI. 24 h after transfection, the cells were imaged in culture medium at 37° C. and 5% CO2 atmophere using a spinning disk setup as described (Bindels et al., 2016). Briefly this microscope system consisted of a Nikon Eclipse Ti-E microscope equipped a 561 nm laser and a Yokogawa CSU X-1 spinning disk unit (operating at 5,000 r.p.m.). The excitation light was directed to the sample via a custom-made dichroic mirror 405/488/561/640 through a 40×CFI Plan Apochromat NA 0.95 air objective (Nikon). The red fluorescence was filtered with a 585-675 nm bandpass (FF01-5121630-25 m, Semrock). Images were recorded with an iXon 897 EMCCD camera (Andor).

Oligomerization Analysis with OSER Method

mScarlet-CytERM-N17, mScarlet-I-CytERM-N17, TagRFP-T-CytERM-N17 and mCherry-CytERM-N17 were cloned as described in (Bindels et al., 2016). FusionRed-CytERM-N17, mScarlet3-CytERM-N17, mScarlet-I3-CytERM-N17 and mScarlet-I3-NCwt-CytERM-N17 were cloned by digesting mCherry-CytERM-N17 with enzymes AgeI and BsrGI to cut out mCherry and replace it with the other RFPs. Cells were analyzed between 22 h and 24 h after transfection. Imaging was performed with the same microscope setup and transfection conditions as described under localization. To obtain an overview of many cells, 16×16 tile scans were acquired with 10% overlap and automated image stitching in NIS elements software leading to 7040×7040 pixel images corresponding to 1.4×1.4 mm imaged areas in each well. Cells were analyzed as described in (Costantini et al., 2012). ROIs of representative cells were assembled for FIG. 14.

### Cytotoxicity

On day 0, 2.5 μg of pmScarlet-pDx, pmScarlet3-pDx, pmScarlet-I3-pDx, pmCherry-pDx or pmFusionRed-pDx was transfected into HeLa cells cultured in T25 flasks, After approximately 4 h, the medium was refreshed. Cells were cultured as described under general methods. Cells were passaged at day 2. At day 2 trypsinized control mEGFP cells were mixed with each of the RFP transfected cells. From the 5 resulting cell mixtures, 4-5 biological replicates were subsequently grown in T25 flasks and passaged on day 4 and on day 6. During passaging on day 2, 4 and 6, two aliquots of (mixed) trypsinized cells were transferred to glass-bottomed 24-well plates supplemented with 0.5 ml culture medium. After 4-5 h of incubation at at 37° C. under 7% humidified C02 atmosphere, the 24-well plates were analyzed for the number of cells displaying green and red fluorescence by fluorescence microscopy, amounting to 8-10 red/green cell number ratios per time point per RFP. For microscopy, the same setup was used as described for the cellular brightness in mammalian cells. For GFP detection a 470 nm LED and for RFP detection a 555 nm LED (SpectraX, Lumencor) was used. The excitation light from these LEDs was passed through 470/24 or 550/15 nm bandpasses, respectively. A quad band cube (MXU 71640, Nikon) was used. Emission was additionally filtered with a 527/70 or 593/46 nm bandpass (both from Semrock) placed in an optical filter changer (Lambda 10-B, Sutter instrument). For the green and red detected channels, the effective excitation and emission bands were green: 458-483 nm excitation, 492-541 nm emission, and red: 543-558 nm excitation and 570-616 nm emission. A 10×CFI Plan Apochromat NA 045 (Nikon MRD00105) objective was used. Images were acquired on an ORCA-Flash4.0 V2 Digital CMOS camera (C11440-22CU, Hamamatsu Photonics). For each well a 12×12 tile of Images of 512×511 pixels each were acquired using a central ROI of 1024×1022 pixels with 2×2 binning and automated image stitching with 10% overlap, resulting in a final image size of 2 channels of 5300×5300 pixels corresponding to a 6.84×6.84 mm imaged area in each well, LED power was 50% and 20% for 470 nm and 555 nm LEDs for the green and red images, respectively. Integration time per image was 100 ins, for each exposure. Between 100 and 3000 fluorescent cells were counted per well for each color using an automated ImageJ macro. Briefly this macro first reduced noise, performed a background correction and applied a threshold intensity after which individual cells were automatically counted using the analyze particles ImageJ command. The threshold intensity for the green channel was identical for all analyzed samples, the threshold intensity for the red channel was different for each RFP to account for differences in cellular brightness, but identical for samples from different days. The effective cellular brightness of the different RFPs was determined as described under ‘brightness mammalian cells’ by analyzing HeLa cells 48 h after transfection with pDress constructs driving expression of mTurquoise2 and the respective RFPs in a 1:1 ratio. The relative cellular brightness and RFP thresholds were 1.307 for mScarlet, 1.899 for mScarlet3, 1.872 for mScarlet-I3, 0.331 for mCherry and 0.298 for FusionRed. This resulted in final thresholds of 24 counts for cells expressing SFP and 100 times the above relative cellular brightness values for the RFP channel thresholds. For each sample (RFP mixture, time point and replicate) the ratio of red fluorescent cells over green fluorescent cells was calculated and displayed.

TABLE IV
mScarlet, and variants thereof

| | AA substitutions relative to | | |
|---|---|---|---|
| mScarlet and variants | mScarlet | AA sequence | DNA sequence |
| mScarlet | — | SEQ ID NO: 1 | SEQ ID NO: 2 |
| mScarlet-i | T74I | SEQ ID NO: 3 | SEQ ID NO: 4 |
| mScarlet-2A | V196I, G220A, E219V, T107S, | SEQ ID NO: 5 | SEQ ID NO: 6 |
| | G156V | | |
| mScarlet-i-2A | V196I, G220A, E219V, T107S, | SEQ ID NO: 7 | SEQ ID NO: 8 |
| | G156V, T74I | | |
| mScarlet-2B | G220A, E219V, T107S, G156V | SEQ ID NO: 9 | SEQ ID NO: 10 |
| mScarlet-i-2B | G220A, E219V, T107S, G156V | SEQ ID NO: 11 | SEQ ID NO: 12 |
| | T74I | | |
| mScarlet-2A-84W | V196I, G220A, E219V, T107S, | SEQ ID NO: 13 | SEQ ID NO: 14 |
| | G156V, Y84W | | |
| mScarlet-i-2A-84W | V196I, G220A, E219V, T107S, | SEQ ID NO: 15 | SEQ ID NO: 16 |
| | G156V, Y84W, T74I | | |
| mScarlet-2B-84W | G220A, E219V, T107S, G156V, | SEQ ID NO: 17 | SEQ ID NO: 18 |
| | Y84W | | |
| mScarlet-i-2B-84W | G220A, E219V, T107S, G156V, | SEQ ID NO: 19 | SEQ ID NO: 20 |
| | Y84W, T74I | | |
| mScarlet-2A-84W-R8 | V196I, G220A, E219V, T107S, | SEQ ID NO: 21 | SEQ ID NO: 22 |
| | G156V, Y84W, T109A, K183R | | |
| mScarlet-3A-NCwt | V196I, G220A, E219V, T107S, | SEQ ID NO: 23 | SEQ ID NO: 24 |
| | G156V, Y84W, T109A, K183R, | | |
| | Y194F | | |
| mScarlet-3-NCwt | V196I, G220A, E219V, T107S, | SEQ ID NO: 25 | SEQ ID NO: 26 |
| | G156V, Y84W, T109A, K183R, | | |
| | Y194F, K48R | | |
| mScarlet-i-2A-84W-nt1 | V196I, G220A, E219V, T107S, | SEQ ID NO: 27 | SEQ ID NO: 28 |
| | G156V, Y84W, T74I, N99I, | | |
| | A105T, T128G, K140R, Y194F, | | |
| | K93R | | |
| mScarlet-i3-NCwt | V196I, G220A, E219V, T107S, | SEQ ID NO: 29 | SEQ ID NO: 30 |
| | G156V, Y84W, T74I, N99I, | | |
| | A105T, T128G, K140R, Y194F, | | |
| mScarlet3 | V196I, G220A, E219V, T107S, | SEQ ID NO: 31 | SEQ ID NO: 32 |
| | G156V, Y84W, T109A, K183R, | | |
| | Y194F, K48R, Δ1, V2M, S3D, | | |
| | K4S, G5T, M227S, D228G, | | |
| | E229G, L230S, Δ231-232 | | |
| mScarlet-3A | V196I, G220A, E219V, T107S, | SEQ ID NO: 33 | SEQ ID NO: 34 |
| | G156V, Y84W, T109A, K183R, | | |
| | Y194F, Δ1, V2M, S3D, K4S, G5T, | | |
| | M227S, D228G, E229G, L230S, | | |
| | Δ231-232 | | |
| mScarlet-i3 | V196I, G220A, E219V, T107S, | SEQ ID NO: 35 | SEQ ID NO: 36 |
| | G156V, Y84W, T74I, N99I, | | |
| | A105T, T128G, K140R, Y194F, | | |
| | Δ1, V2M, S3D, K4S, G5T, | | |
| | M227S, D228G, E229G, L230S, | | |
| | Δ231-232 | | |

### REFERENCES

Bajar, B. T., Wang, E. S., Lam, A. J., Kim, B. B., Jacobs, C. L., Howe, E. S., Davidson, M. W., Lin, M. Z., & Chu, J. (2016). Improving brightness and photostability of green and red fluorescent proteins for live cell imaging and FRET reporting. Scientific Reports, 6. https://doi.org/10.1038/srep20889
Bindels, D. S., Haarbosch, L., Van Weeren, L., Postma, M., Wiese, K. E., Mastop, M., Aumonier, S., Gotthard, G., Royant, A., Hink, M. A., & Gadella, T. W. J. (2016). MScarlet: A bright monomeric red fluorescent protein for cellular imaging. Nature Methods, 14(1), https://doi.org/i0.1038/nmeth.4074
Bindels, D. S., Postma, M., Haarbosch, L., van Weeren, L., & Gadella, T. W. J. (2020). Multiparameter screening method for developing optimized red-fluorescent proteins. Nature Protocols, 15(2). https://doi.org/I0.1038/s41596-019-0250-7
Costantini, L. M., Fossati, M., Francolini, M., & Snapp, E. L. (2012). Assessing the Tendency of Fluorescent Proteins to Oligomerize Under Physiologic Conditions. Traffic, 13(5), 643-649. https://doi.org/I0.1111/j.1600-0854.2012.01336.x
Gross, L. A., Baird, G. S., Hoffman, R. C., Baldridge, K. K., & Tsien, R. Y. (2000). The structure of the chromophore within DsRed, a red fluorescent protein from coral. Proceedings of the National Academy of Sciences of the United States of America, 97(22), 11990-11995. https://doi.org/I0.1073/pnas.97.22.11990
Shagin, D. A., Barsova, E. V., Yanushevich, Y. G., Fradkov, A. F., Lukyanov, K. A., Labas, Y. A., Semenova, T. N., Ugalde, J. A., Meyers, A., Nunez, J. M., Widder, E. A., Lukyanov, S. A., & Matz, M. V. (2004). GFP-like Proteins as Ubiquitous Metazoan Superfamily: Evolution of Functional Features and Structural Complexity. Molecular Biology and Evolution, 21(5), 841-850. https://doi.orgi10.1093/molbeV/mshO79
Shaner, N. C., Campbell, R. E., Steinbach, P. A., Giepmans, B. N. G., Palmer, A. E., & Tsien, R. Y. (2004). Improved monomeric red, orange and yellow fluorescent proteins derived from Discosoma sp. red fluorescent protein. Nature Biotechnology, 22(12), 1567-1572. https://doi.org/10.1038/nbt1037
Shaner, N. C., Lin, M. Z., McKeown, M. R., Steinbach, P. A., Hazelwood, K. L., Davidson, M. W., & Tsien, R. Y. (2008). Improving the photostability of bright monomeric orange and red fluorescent proteins, Nature Methods, 5(6), 545-551, https://doi.org/10.1038/nmeth.1209
Shcherbo, D., Murphy, C. S., Ermakova, G. V., Solovieva, E. A., Chepurnykh, T. V., Shcheglov A. S., Verkhusha, V. V., Pletnev, V. Z., Hazelwood, K. L., Roche. P. M., Lukyanov. S., Zaraisky, A. G., Davidson, M. W., & Chudakov, D. M. (2009). Far-red fluorescent tags for protein imaging in living tissues. Biochemical Journal, 418(3), 567-574. https://doi.org/10.1042/BJ20081949
Shemiakina, I. I., Ermakova, G. V., Cranfill, P. J., Baird, M. A., Evans, R. A., Souslova, E. A., Staroverov, D. B., Gorokhovatsky, A. Y., Putintseva, E. V., Gorodnicheva, T. V., Chepurnykh, T. V., Strukova, L., Lukyanov, S., Zaraisky, A. G., Davidson, M. W., Chudakov, D. M., & Shcherbo, D. (2012). A monomeric red fluorescent protein with low cytotoxicity. Nature Communications, 3. https://doi.org/I0.1038/ncomms2208
Valbuena, F., Fizgerald, I., Strack, R. L., Andruska, N., Smith, L., & Glick, B. S. (2019), A photostable monomeric superfolder GFP. https://doi.org/i0.1101/811588
Vermeer, J. E. M., Van Munster, E. B., Vischer, N. O., & Gadella Jr., T. W. J. (2004). Probing plasma membrane microdomains in cowpea protoplasts using lipidated GFP-fusion proteins and multimode FRET microscopy. Journal of Microscopy, 214(2). https://doi.org/10.1111/j.0022-2720.2004.01318.x

1. A variant of monomeric Scarlet Red Fluorescent protein (mScarlet RFP), wherein the variant comprises amino acid substitution G220A, or a combination of amino acid substitutions G220A and V196I, and optionally amino acid substitution T74I, when compared to the amino acid sequence of the mScarlet RFP depicted in SEQ ID NO:1, and wherein the amino acid sequence of the variant has a sequence identity of at least 80% with SEQ ID NO:1.

2. A variant according to claim 1, wherein the variant further comprises amino acid substitution E219V.

3. A variant according to claim 1, wherein the variant further comprises amino acid substitutions T107S and G156V.

4. A variant according to claim 1, wherein the variant further comprises amino acid substitution Y84W.

5. A variant according to claim 1, wherein the said-variant further comprises amino acid substitution Y194F.

6. A variant according to claim 1, wherein the variant lacks amino acid substitution T74I and further comprises amino acid substitutions T109A and K183R.

7. A variant according to claim 1, wherein the variant comprises the amino acid substitution T74I, and further amino acid substitutions N99I, A105T, T128G, and K140R.

8. A variant according to claim 6, wherein the variant comprises the amino acid substitutions K48R, Y84W, T107S, T109A, G156V, K183R, Y194F, V196I, E219V, and G220A when compared to the amino acid sequence of the mScarlet RFP depicted in SEQ ID NO:1.

9. A variant according to claim 7, wherein the variant comprises amino acid substitutions T74I, Y84W, N99I, A105T, T107S, T128G, K140R, G156V, Y194F, V196I, E219V and G220A when compared to the amino acid sequence of the mScarlet RFP depicted in SEQ ID NO:1.

10. A variant according to claim 1, the variant additionally comprising amino acid substitutions and deletions ΔM1, V2M, S3D, K4S, G5T and M227S, D228G, E229G, L230S, ΔY231, ΔK232.

11. A variant according to claim 1, the variant comprising an amino acid sequence selected from the group consisting of amino acid sequences depicted in SEQ ID NO's 5, 7, 9, 11, 13, 15, 17, 19, 21, 23, 25, 27, 29, 31, 33, and 35.

12. A nucleic acid sequence encoding a variant according to claim 1.

13. A nucleic acid according to claim 12, wherein the nucleic acid comprises a nucleic acid sequence selected from the group of nucleic acid sequences depicted in SEQ ID NO's 6, 8, 10, 12, 14, 16, 18, 20, 22, 24, 26, 28, 30, 32, 34, and 36.

14. A nucleic acid according to claim 13, wherein the nucleic acid has the nucleic acid sequences depicted in SEQ ID NO: 32 or 36.

15. An expression vector comprising the nucleic acid according to claim 12.

16. A host cell comprising the expression vector of claim 15.


SEQUENCE LISTING (43 sequences)

SEQ ID NO: 1 - protein, 232 aa, Artificial Sequence
  synthetic construct
MVSKGEAVIKEFMRFKVHMEGSMNGHEFEIEGEGEGRPYEGTQTAKLKVTKGGPLPFSWDILSPQFMYGSRAFTKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHSTGGMDELYK

SEQ ID NO: 2 - DNA, 696 bp, Artificial Sequence
  synthetic construct
atggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcaccaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcatccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaag

SEQ ID NO: 3 - protein, 232 aa, Artificial Sequence
  synthetic construct
MVSKGEAVIKEFMRFKVHMEGSMNGHEFEIEGEGEGRPYEGTQTAKLKVTKGGPLPFSWDILSPQFMYGSRAFIKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHSTGGMDELYK

SEQ ID NO: 4 - DNA, 696 bp, Artificial Sequence
  synthetic construct
atggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcatccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaag

SEQ ID NO: 5 - protein, 232 aa, Artificial Sequence
  synthetic construct
MVSKGEAVIKEFMRFKVHMEGSMNGHEFEIEGEGEGRPYEGTQTAKLKVTKGGPLPFSWDILSPQFMYGSRAFTKHPADIPDYYKQSFPEGFKWERVMNFEDGGAVSVTQDTSLEDGTLIYKVKLRGTNFPPDGPVMQKKTMGWEASTERLYPEDVVLKGDIKMALRLKDGGRYLADFKTTYKAKKPVQMPGAYNIDRKLDITSHNEDYTVVEQYERSVARHSTGGMDELYK

SEQ ID NO: 6 - DNA, 696 bp, Artificial Sequence
  synthetic construct
atggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcaccaagcaccccgccgacatccccgactactataagcagtccttccccgagggcttcaagtgggagcgcgtgatgaacttcgaggacggcggcgccgtgtccgtgacccaggacacctccctggaggacggcaccctgatctacaaggtgaagctccgcggcaccaacttccctcctgacggccccgtaatgcagaagaagacaatgggctgggaagcatccaccgagcggttgtaccccgaggacgtcgtgctgaagggcgacattaagatggccctgcgcctgaaggacggcggccgctacctggcggacttcaagaccacctacaaggccaagaagcccgtgcagatgcccggcgcctacaacatcgaccgcaagttggacatcacctcccacaacgaggactacaccgtggtggaacagtacgaacgctccgtggcccgccactccaccggcggcatggacgagctgtacaag

SEQ ID NO: 7 - protein, 232 aa, Artificial Sequence
  synthetic construct
MVSKGEAVIKEFMRFKVHMEGSMNGHEFEIEGEGEGRPYEGTQTAKLKVTKGGPLPFSWDILSPQFMYGSRAFIKHPADIPDYYKQSFPEGFKWERVMNFEDGGAVSVTQDTSLEDGTLIYKVKLRGTNFPPDGPVMQKKTMGWEASTERLYPEDVVLKGDIKMALRLKDGGRYLADFKTTYKAKKPVQMPGAYNIDRKLDITSHNEDYTVVEQYERSVARHSTGGMDELYK

SEQ ID NO: 8 - DNA, 696 bp, Artificial Sequence
  synthetic construct
atggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgtccgtgacccaggacacctccctggaggacggcaccctgatctacaaggtgaagctccgcggcaccaacttccctcctgacggccccgtaatgcagaagaagacaatgggctgggaagcatccaccgagcggttgtaccccgaggacgtcgtgctgaagggcgacattaagatggccctgcgcctgaaggacggcggccgctacctggcggacttcaagaccacctacaaggccaagaagcccgtgcagatgcccggcgcctacaacatcgaccgcaagttggacatcacctcccacaacgaggactacaccgtggtggaacagtacgaacgctccgtggcccgccactccaccggcggcatggacgagctgtacaag

SEQ ID NO: 9 - protein, 232 aa, Artificial Sequence
  synthetic construct
MVSKGEAVIKEFMRFKVHMEGSMNGHEFEIEGEGEGRPYEGTQTAKLKVTKGGPLPFSWDILSPQFMYGSRAFTKHPADIPDYYKQSFPEGFKWERVMNFEDGGAVSVTQDTSLEDGTLIYKVKLRGTNFPPDGPVMQKKTMGWEASTERLYPEDVVLKGDIKMALRLKDGGRYLADFKTTYKAKKPVQMPGAYNVDRKLDITSHNEDYTVVEQYERSVARHSTGGMDELYK

SEQ ID NO: 10 - DNA, 696 bp, Artificial Sequence
  synthetic construct
atggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcaccaagcaccccgccgacatccccgactactataagcagtccttccccgagggcttcaagtgggagcgcgtgatgaacttcgaggacggcggcgccgtgtccgtgacccaggacacctccctggaggacggcaccctgatctacaaggtgaagctccgcggcaccaacttccctcctgacggccccgtaatgcagaagaagacaatgggctgggaagcatccaccgagcggttgtaccccgaggacgtcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgtggcccgccactccaccggcggcatggacgagctgtacaag

SEQ ID NO: 11 - protein, 232 aa, Artificial Sequence
  synthetic construct
MVSKGEAVIKEFMRFKVHMEGSMNGHEFEIEGEGEGRPYEGTQTAKLKVTKGGPLPFSWDILSPQFMYGSRAFIKHPADIPDYYKQSFPEGFKWERVMNFEDGGAVSVTQDTSLEDGTLIYKVKLRGTNFPPDGPVMQKKTMGWEASTERLYPEDVVLKGDIKMALRLKDGGRYLADFKTTYKAKKPVQMPGAYNVDRKLDITSHNEDYTVVEQYERSVARHSTGGMDELYK

SEQ ID NO: 12 - DNA, 696 bp, Artificial Sequence
  synthetic construct
atggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgtccgtgacccaggacacctccctggaggacggcaccctgatctacaaggtgaagctccgcggcaccaacttccctcctgacggccccgtaatgcagaagaagacaatgggctgggaagcatccaccgagcggttgtaccccgaggacgtcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgtggcccgccactccaccggcggcatggacgagctgtacaag

SEQ ID NO: 13 - protein, 232 aa, Artificial Sequence
  synthetic construct
MVSKGEAVIKEFMRFKVHMEGSMNGHEFEIEGEGEGRPYEGTQTAKLKVTKGGPLPFSWDILSPQFMYGSRAFTKHPADIPDYWKQSFPEGFKWERVMNFEDGGAVSVTQDTSLEDGTLIYKVKLRGTNFPPDGPVMQKKTMGWEASTERLYPEDVVLKGDIKMALRLKDGGRYLADFKTTYKAKKPVQMPGAYNIDRKLDITSHNEDYTVVEQYERSVARHSTGGMDELYK

SEQ ID NO: 14 - DNA, 696 bp, Artificial Sequence
  synthetic construct
atggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcaccaagcaccccgccgacatccccgactactggaagcagtccttccccgagggcttcaagtgggagcgcgtgatgaacttcgaggacggcggcgccgtgtccgtgacccaggacacctccctggaggacggcaccctgatctacaaggtgaagctccgcggcaccaacttccctcctgacggccccgtaatgcagaagaagacaatgggctgggaagcatccaccgagcggttgtaccccgaggacgtcgtgctgaagggcgacattaagatggccctgcgcctgaaggacggcggccgctacctggcggacttcaagaccacctacaaggccaagaagcccgtgcagatgcccggcgcctacaacatcgaccgcaagttggacatcacctcccacaacgaggactacaccgtggtggaacagtacgaacgctccgtggcccgccactccaccggcggcatggacgagctgtacaag

SEQ ID NO: 15 - protein, 232 aa, Artificial Sequence
  synthetic construct
MVSKGEAVIKEFMRFKVHMEGSMNGHEFEIEGEGEGRPYEGTQTAKLKVTKGGPLPFSWDILSPQFMYGSRAFIKHPADIPDYWKQSFPEGFKWERVMNFEDGGAVSVTQDTSLEDGTLIYKVKLRGTNFPPDGPVMQKKTMGWEASTERLYPEDVVLKGDIKMALRLKDGGRYLADFKTTYKAKKPVQMPGAYNIDRKLDITSHNEDYTVVEQYERSVARHSTGGMDELYK

SEQ ID NO: 16 - DNA, 696 bp, Artificial Sequence
  synthetic construct
atggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactggaagcagtccttccccgagggcttcaagtgggagcgcgtgatgaacttcgaggacggcggcgccgtgtccgtgacccaggacacctccctggaggacggcaccctgatctacaaggtgaagctccgcggcaccaacttccctcctgacggccccgtaatgcagaagaagacaatgggctgggaagcatccaccgagcggttgtaccccgaggacgtcgtgctgaagggcgacattaagatggccctgcgcctgaaggacggcggccgctacctggcggacttcaagaccacctacaaggccaagaagcccgtgcagatgcccggcgcctacaacatcgaccgcaagttggacatcacctcccacaacgaggactacaccgtggtggaacagtacgaacgctccgtggcccgccactccaccggcggcatggacgagctgtacaag

SEQ ID NO: 17 - protein, 232 aa, Artificial Sequence
  synthetic construct
MVSKGEAVIKEFMRFKVHMEGSMNGHEFEIEGEGEGRPYEGTQTAKLKVTKGGPLPFSWDILSPQFMYGSRAFTKHPADIPDYWKQSFPEGFKWERVMNFEDGGAVSVTQDTSLEDGTLIYKVKLRGTNFPPDGPVMQKKTMGWEASTERLYPEDVVLKGDIKMALRLKDGGRYLADFKTTYKAKKPVQMPGAYNVDRKLDITSHNEDYTVVEQYERSVARHSTGGMDELYK

SEQ ID NO: 18 - DNA, 696 bp, Artificial Sequence
  synthetic construct
atggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcaccaagcaccccgccgacatccccgactactggaagcagtccttccccgagggcttcaagtgggagcgcgtgatgaacttcgaggacggcggcgccgtgtccgtgacccaggacacctccctggaggacggcaccctgatctacaaggtgaagctccgcggcaccaacttccctcctgacggccccgtaatgcagaagaagacaatgggctgggaagcatccaccgagcggttgtaccccgaggacgtcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgtggcccgccactccaccggcggcatggacgagctgtacaag

SEQ ID NO: 19 - protein, 232 aa, Artificial Sequence
  synthetic construct
MVSKGEAVIKEFMRFKVHMEGSMNGHEFEIEGEGEGRPYEGTQTAKLKVTKGGPLPFSWDILSPQFMYGSRAFIKHPADIPDYWKQSFPEGFKWERVMNFEDGGAVSVTQDTSLEDGTLIYKVKLRGTNFPPDGPVMQKKTMGWEASTERLYPEDVVLKGDIKMALRLKDGGRYLADFKTTYKAKKPVQMPGAYNVDRKLDITSHNEDYTVVEQYERSVARHSTGGMDELYK

SEQ ID NO: 20 - DNA, 696 bp, Artificial Sequence
  synthetic construct
atggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactggaagcagtccttccccgagggcttcaagtgggagcgcgtgatgaacttcgaggacggcggcgccgtgtccgtgacccaggacacctccctggaggacggcaccctgatctacaaggtgaagctccgcggcaccaacttccctcctgacggccccgtaatgcagaagaagacaatgggctgggaagcatccaccgagcggttgtaccccgaggacgtcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgtggcccgccactccaccggcggcatggacgagctgtacaag

SEQ ID NO: 21 - protein, 232 aa, Artificial Sequence
  synthetic construct
MVSKGEAVIKEFMRFKVHMEGSMNGHEFEIEGEGEGRPYEGTQTAKLKVTKGGPLPFSWDILSPQFMYGSRAFTKHPADIPDYWKQSFPEGFKWERVMNFEDGGAVSVAQDTSLEDGTLIYKVKLRGTNFPPDGPVMQKKTMGWEASTERLYPEDVVLKGDIKMALRLKDGGRYLADFKTTYRAKKPVQMPGAYNIDRKLDITSHNEDYTVVEQYERSVARHSTGGMDELYK

SEQ ID NO: 22 - DNA, 696 bp, Artificial Sequence
  synthetic construct
atggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcacgaagcaccccgccgacatccccgactactggaagcagtccttccccgagggcttcaagtgggagcgcgtgatgaacttcgaggacggcggcgccgtgtccgtggcccaggacacctccctggaggacggcaccctgatctacaaggtgaagctccgcggcaccaacttccctcctgacggccccgtaatgcagaagaagacaatgggctgggaagcatccaccgagcggttgtaccccgaggacgtcgtgctgaagggcgacattaagatggccctgcgcctgaaggacggcggccgctacctggcggacttcaagaccacctacagggccaagaagcccgtgcagatgcccggcgcctacaacatcgaccgcaagttggacatcacatcccacaacgaggactacaccgtggtggaacagtacgaacgctccgtggcccgccactccaccggcggcatggacgagctgtacaag

SEQ ID NO: 23 - protein, 232 aa, Artificial Sequence
  synthetic construct
MVSKGEAVIKEFMRFKVHMEGSMNGHEFEIEGEGEGRPYEGTQTAKLKVTKGGPLPFSWDILSPQFMYGSRAFTKHPADIPDYWKQSFPEGFKWERVMNFEDGGAVSVAQDTSLEDGTLIYKVKLRGTNFPPDGPVMQKKTMGWEASTERLYPEDVVLKGDIKMALRLKDGGRYLADFKTTYRAKKPVQMPGAFNIDRKLDITSHNEDYTVVEQYERSVARHSTGGMDELYK

SEQ ID NO: 24 - DNA, 696 bp, Artificial Sequence
  synthetic construct
atggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcaccaagcaccccgccgacatccccgactactggaagcagtccttccccgagggcttcaagtgggagcgcgtgatgaacttcgaggacggcggcgccgtgtccgtggcccaggacacctccctggaggacggcaccctgatctacaaggtgaagctccgcggcaccaacttccctcctgacggccccgtaatgcagaagaagacaatgggctgggaagcatccaccgagcggttgtaccccgaggacgtcgtgctgaagggcgacattaagatggccctgcgcctgaaggacggcggccgctacctggcggacttcaagaccacctacagggccaagaagcccgtgcagatgcccggcgccttcaacatcgaccgcaagttggacatcacctcccacaacgaggactacaccgtggtggaacagtacgaacgctccgtggcccgccactccaccggcggcatggacgagctgtacaag

SEQ ID NO: 25 - protein, 232 aa, Artificial Sequence
  synthetic construct
MVSKGEAVIKEFMRFKVHMEGSMNGHEFEIEGEGEGRPYEGTQTAKLRVTKGGPLPFSWDILSPQFMYGSRAFTKHPADIPDYWKQSFPEGFKWERVMNFEDGGAVSVAQDTSLEDGTLIYKVKLRGTNFPPDGPVMQKKTMGWEASTERLYPEDVVLKGDIKMALRLKDGGRYLADFKTTYRAKKPVQMPGAFNIDRKLDITSHNEDYTVVEQYERSVARHSTGGMDELYK

SEQ ID NO: 26 - DNA, 696 bp, Artificial Sequence
  synthetic construct
atggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgagggtgaccaagggtggccccctgcccttctcctgggacatcctgtcccctcagttcatgtacggctccagggccttcaccaagcaccccgccgacatccccgactactggaagcagtccttccccgagggcttcaagtgggagcgcgtgatgaacttcgaggacggcggcgccgtgtccgtggcccaggacacctccctggaggacggcaccctgatctacaaggtgaagctccgcggcaccaacttccctcctgacggccccgtaatgcagaagaagacaatgggctgggaagcatccaccgagcggttgtaccccgaggacgtcgtgctgaagggcgacattaagatggccctgcgcctgaaggacggcggccgctacctggcggacttcaagaccacctacagggccaagaagcccgtgcagatgcccggcgccttcaacatcgaccgcaagttggacatcacctcccacaacgaggactacaccgtggtggaacagtacgaacgctccgtggcccgccactccaccggcggcatggacgagctgtacaag

SEQ ID NO: 27 - protein, 232 aa, Artificial Sequence
  synthetic construct
MVSKGEAVIKEFMRFKVHMEGSMNGHEFEIEGEGEGRPYEGTQTAKLKVTKGGPLPFSWDILSPQFMYGSRAFIKHPADIPDYWKQSFPEGFRWERVMIFEDGGTVSVTQDTSLEDGTLIYKVKLRGGNFPPDGPVMQKRTMGWEASTERLYPEDVVLKGDIKMALRLKDGGRYLADFKTTYKAKKPVQMPGAFNIDRKLDITSHNEDYTVVEQYERSVARHSTGGMDELYK

SEQ ID NO: 28 - DNA, 696 bp, Artificial Sequence
  synthetic construct
atggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactggaagcagtccttccccgagggcttcaggtgggagcgcgtgatgatcttcgaggacggcggcaccgtgtccgtgacccaggacacctccctggaggacggcaccctgatctacaaggtgaagctccgcggcggcaacttccctcctgacggccccgtaatgcagaagaggacaatgggctgggaagcatccaccgagcggttgtaccccgaggacgtcgtgctgaagggcgacattaagatggccctgcgcctgaaggacggcggccgctacctggcggacttcaagaccacctacaaggccaagaagcccgtgcagatgcccggcgccttcaacatcgaccgcaagttggacatcacctcccacaacgaggactacaccgtggtggaacagtacgaacgctccgtggcccgccactccaccggcggcatggacgagctgtacaag

SEQ ID NO: 29 - protein, 232 aa, Artificial Sequence
  synthetic construct
MVSKGEAVIKEFMRFKVHMEGSMNGHEFEIEGEGEGRPYEGTQTAKLKVTKGGPLPFSWDILSPQFMYGSRAFIKHPADIPDYWKQSFPEGFKWERVMIFEDGGTVSVTQDTSLEDGTLIYKVKLRGGNFPPDGPVMQKRTMGWEASTERLYPEDVVLKGDIKMALRLKDGGRYLADFKTTYKAKKPVQMPGAFNIDRKLDITSHNEDYTVVEQYERSVARHSTGGMDELYK

SEQ ID NO: 30 - DNA, 696 bp, Artificial Sequence
  synthetic construct
atggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactggaagcagtccttccccgagggcttcaagtgggagcgcgtgatgatcttcgaggacggcggcaccgtgtccgtgacccaggacacctccctggaggacggcaccctgatctacaaggtgaagctccgcggcggcaacttccctcctgacggccccgtaatgcagaagaggacaatgggctgggaagcatccaccgagcggttgtaccccgaggacgtcgtgctgaagggcgacattaagatggccctgcgcctgaaggacggcggccgctacctggcggacttcaagaccacctacaaggccaagaagcccgtgcagatgcccggcgccttcaacatcgaccgcaagttggacatcacctcccacaacgaggactacaccgtggtggaacagtacgaacgctccgtggcccgccactccaccggcggcatggacgagctgtacaag

SEQ ID NO: 31 - protein, 229 aa, Artificial Sequence
  synthetic construct
MDSTEAVIKEFMRFKVHMEGSMNGHEFEIEGEGEGRPYEGTQTAKLRVTKGGPLPFSWDILSPQFMYGSRAFTKHPADIPDYWKQSFPEGFKWERVMNFEDGGAVSVAQDTSLEDGTLIYKVKLRGTNFPPDGPVMQKKTMGWEASTERLYPEDVVLKGDIKMALRLKDGGRYLADFKTTYRAKKPVQMPGAFNIDRKLDITSHNEDYTVVEQYERSVARHSTGGSGGS

SEQ ID NO: 32 - DNA, 687 bp, Artificial Sequence
  synthetic construct
atggatagcaccgaggcagtgatcaaggagttcatgcggttcaaggtgcacatggagggctccatgaacggccacgagttcgagatcgagggcgagggcgagggccgcccctacgagggcacccagaccgccaagctgagggtgaccaagggtggccccctgcccttctcctgggacatcctgtcccctcagttcatgtacggctccagggccttcacgaagcaccccgccgacatccccgactactggaagcagtccttccccgagggcttcaagtgggagcgcgtgatgaacttcgaggacggcggcgccgtgtccgtggcccaggacacctccctggaggacggcaccctgatctacaaggtgaagctccgcggcaccaacttccctcctgacggccccgtaatgcagaagaagacaatgggctgggaagcatccaccgagcggttgtaccccgaggacgtcgtgctgaagggcgacattaagatggccctgcgcctgaaggacggcggccgctacctggcggacttcaagaccacctacagggccaagaagcccgtgcagatgcccggcgccttcaacatcgaccgcaagttggacatcacatcccacaacgaggactacaccgtggtggaacagtacgaacgctccgtggcccgccactccaccggcggctccggtggctcc

SEQ ID NO: 33 - protein, 229 aa, Artificial Sequence
  synthetic construct
MDSTEAVIKEFMRFKVHMEGSMNGHEFEIEGEGEGRPYEGTQTAKLKVTKGGPLPFSWDILSPQFMYGSRAFTKHPADIPDYWKQSFPEGFKWERVMNFEDGGAVSVAQDTSLEDGTLIYKVKLRGTNFPPDGPVMQKKTMGWEASTERLYPEDVVLKGDIKMALRLKDGGRYLADFKTTYRAKKPVQMPGAFNIDRKLDITSHNEDYTVVEQYERSVARHSTGGSGGS

SEQ ID NO: 34 - DNA, 687 bp, Artificial Sequence
  synthetic construct
atggatagcaccgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcaccaagcaccccgccgacatccccgactactggaagcagtccttccccgagggcttcaagtgggagcgcgtgatgaacttcgaggacggcggcgccgtgtccgtggcccaggacacctccctggaggacggcaccctgatctacaaggtgaagctccgcggcaccaacttccctcctgacggccccgtaatgcagaagaagacaatgggctgggaagcatccaccgagcggttgtaccccgaggacgtcgtgctgaagggcgacattaagatggccctgcgcctgaaggacggcggccgctacctggcggacttcaagaccacctacagggccaagaagcccgtgcagatgcccggcgccttcaacatcgaccgcaagttggacatcacctcccacaacgaggactacaccgtggtggaacagtacgaacgctccgtggcccgccactccaccggcggctccggtggctcc

SEQ ID NO: 35 - protein, 229 aa, Artificial Sequence
  synthetic construct
MDSTEAVIKEFMRFKVHMEGSMNGHEFEIEGEGEGRPYEGTQTAKLKVTKGGPLPFSWDILSPQFMYGSRAFIKHPADIPDYWKQSFPEGFKWERVMIFEDGGTVSVTQDTSLEDGTLIYKVKLRGGNFPPDGPVMQKRTMGWEASTERLYPEDVVLKGDIKMALRLKDGGRYLADFKTTYKAKKPVQMPGAFNIDRKLDITSHNEDYTVVEQYERSVARHSTGGSGGS

SEQ ID NO: 36 - DNA, 687 bp, Artificial Sequence
  synthetic construct
atggatagcaccgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactggaagcagtccttccccgagggcttcaagtgggagcgcgtgatgatcttcgaggacggcggcaccgtgtccgtgacccaggacacctccctggaggacggcaccctgatctacaaggtgaagctccgcggcggcaacttccctcctgacggccccgtaatgcagaagaggacaatgggctgggaagcatccaccgagcggttgtaccccgaggacgtcgtgctgaagggcgacattaagatggccctgcgcctgaaggacggcggccgctacctggcggacttcaagaccacctacaaggccaagaagcccgtgcagatgcccggcgccttcaacatcgaccgcaagttggacatcacctcccacaacgaggactacaccgtggtggaacagtacgaacgctccgtggcccgccactccaccggcggctccggtggctcc

SEQ ID NO: 37 - protein, 33 aa, Artificial Sequence
  linker sequence
QYERSVARHSTGGSGGSLYKVSKGEELFTGVVP

SEQ ID NO: 38 - protein, 33 aa, Artificial Sequence; identical to SEQ ID NO: 37
  linker sequence

SEQ ID NO: 39 - protein, 33 aa, Artificial Sequence
  linker sequence
QYERSEGRHSTGGMDELYKMVSKGEELFTGVVP

SEQ ID NO: 40 - protein, 33 aa, Artificial Sequence; identical to SEQ ID NO: 39
  linker sequence

SEQ ID NO: 41 - protein, 33 aa, Artificial Sequence
  linker sequence
QHEVAVARYSTGGAGDLYKMVSKGEELFTGVVP

SEQ ID NO: 42 - protein, 33 aa, Artificial Sequence
  linker sequence
QYERAEGRHSTGGMDELYKMVSKGEELFTGVVP

SEQ ID NO: 43 - protein, 33 aa, Artificial Sequence
  linker sequence
QREVAVAKYSNLGMDELYKMVSKGEELFTGVVP